(19) United States
(12) Patent Application Publication
(10) Pub. No.: US 2022/0273607 A1
(43) Pub. Date: Sep. 1, 2022

(54) METHODS OF USING MODIFIED CYTOTOXINS TO TREAT CANCER

(71) Applicant: NORTHWESTERN UNIVERSITY, Evanston, IL (US)

(72) Inventor: Nathan C. GIANNESCHI, Evanston, IL (US)

(73) Assignee: NORTHWESTERN UNIVERSITY, Evanston, IL (US)

(21) Appl. No.: 17/625,023

(22) PCT Filed: Jul. 8, 2020
(86) PCT No.: PCT/US2020/041204

§ 371 Date: Jan. 5, 2022

Related U.S. Application Data
(60) Provisional application No. 62/872,149, filed on Jul. 9, 2019.

(51) Int. Cl. A61K 31/337; A61K 47/54; A61K 47/42; A61K 45/06; A61K 39/395; A61P 35/00

(52) U.S. Cl. CPC A61K 31/337; A61K 47/542; A61K 47/42; A61K 45/06; A61K 39/3955; A61P 35/00

(57) ABSTRACT

The present disclosure provides methods of using prodrugs of small-molecule cytotoxins for the treatment of cancer. In some embodiments, the cancer is a tumor comprising cells that overexpress fatty acid uptake proteins, such as cells that overexpress fatty acid translocase CD36. In some other aspects, the disclosure provides compositions suitable for use in such methods. In some further aspects, the disclosure provides combination therapies that may be suitable used in combination with the use of small-molecule prodrugs disclosed herein.

### CROSS-REFERENCE TO RELATED APPLICATIONS

The present application claims priority to U.S. Provisional Application No. 62/872,149, filed Jul. 9, 2019, which is hereby incorporated by reference as though set forth herein in its entirety.

### TECHNICAL FIELD

The present disclosure provides methods of using prodrugs of small-molecule cytotoxins for the treatment of cancer. In some embodiments, the cancer is a tumor comprising cells that overexpress fatty acid uptake proteins, such as cells that overexpress fatty acid translocase CD36 (“CD36”). In some other aspects, the disclosure provides compositions suitable for use in such methods. In some further aspects, the disclosure provides combination therapies that may be suitable used in combination with the use of small-molecule prodrugs disclosed herein.

### DESCRIPTION OF RELATED ART

Cancer refers to a group of diseases characterized by the formation of malignant tumors or neoplasms, which involve abnormal cell growth and have the potential to invade adjacent tissue and spread to other parts of the body. There are more than 14 million new diagnoses of cancer annually. Moreover, cancer accounts for more than 8 million deaths each year, which is about 15% of all deaths worldwide. In developed countries, cancer accounts for an even higher percentage of deaths.

Therapies for cancer have improved significantly over the years. In particular, an increasing number of cytotoxic agents have been discovered. These agents generally work by killing the cancer cells. But cytotoxic agents can be harmful to normal cells as well. Therefore, subjects undergoing treatment with such agents often suffer certain side-effects from the treatment. In some cases, the side-effects pose such a substantial risk that it may be necessary to administer very limited quantities of cytotoxic agents. So, while there is a general desire to discover increasingly toxic chemotherapeutic agents, it is also desirable to develop new means of directing those compounds selectively to cancer cells and away from normal cells.

Various strategies have been used to assist in directing chemotherapeutic agents selectively to cancer cells. For example, certain compounds rely on passive targeting, where the compound is selectively directed toward cancer cells (e.g., in a solid tumor) as a result of the fact that cancer cells tend to divide more rapidly than other cells and therefore have a higher appetite for certain biological building blocks. For example, gemcitabine, a commonly used cytotoxin, contains a sugar-like moiety as well as a toxic payload (a 5-fluorouracil moiety). Because cancer cells may have a higher need for sugar than other cells, gemcitabine is passively drawn to rapidly dividing cancer cells because it looks like a sugar molecule. Once at the cancer cell, gemcitabine can release its toxic payload. In some other cases, the targeting may be active, where the cytotoxic agent includes a moiety that binds selectively to a protein that is overexpressed in certain cancer cells. For example, pemetrexed includes a moiety that mimics folic acid, and thereby allows the drug to actively target cancer cells that overexpress folic acid receptors.

It was previously discovered that certain lipid-mimicking cytotoxin prodrugs may be useful for treating cancer. Such compounds were disclosed in WO 2017/053391. In certain preclinical studies using standard cancer xenograft tumors, the compounds were demonstrated to be effective at reducing tumor size. It was also demonstrated that the preclinical subjects (mice) were able to withstand higher doses of the cytotoxin than observed with comparable commercially available cytotoxin formulations.

Even so, it was not understood how such compounds may be effective, and for what types of cancer they may be most beneficially used as treatments. Therefore, there is a continuing need to discover in what contexts such compounds may be most beneficially employed.

### SUMMARY

The present disclosure provides improved methods of using the cytotoxins set forth in WO 2017/053391. In particular, it was discovered that these prodrugs are selectively taken up by cancer cells that overexpress certain fatty acid uptake proteins, such as cells that overexpress CD36. Thus, it was discovered that the prodrug feature of these compounds did not merely improve non-covalent binding to human serum albumin (HSA) and thereby decrease off-target damage to healthy tissue. But the prodrug feature also allowed the prodrug to be taken up selectively by certain cancer cells via the overexpression of surface proteins that mediate the transport of fatty acids to the interior of the cell. Thus, the prodrug feature permits active targeting of particular types of cancer that characteristically overexpress fatty acid uptake proteins, such as CD36.

In at least one aspect, the disclosure provides a method of treating cancer comprising administering to a subject in need thereof a compound of formula (I):

A¹-X¹-X²-A² (I)

wherein: A¹ is a carboxylic acid group, a carboxylate anion, or a carboxylate ester; A² is a cytotoxic drug moiety, which has a molecular weight of no more than 1600 Da; X¹ is a hydrophobic group; and X² is a direct bond, an organic group, or a heteroatom group selected from the group consisting of —O—, —S—, —S(═O)—, —S(═O)₂—, —S—S—, —N═, ═N—, —N(H)—, —N═N—N(H)—, —N(H)—N═N—, —N(OH)—, or —N(═O)—; and wherein the cancer comprises cells that overexpress one or more fatty acid uptake proteins. In some embodiments, the cytotoxic drug moiety is a taxane moiety, such as a paclitaxel moiety. In some embodiments, the hydrophobic group is a C₁₂₋₂₂ hydrocarbylene group, which is optionally substituted. In some embodiments, X² is an organic group, such as a carbonyl group, i.e., —C(═O)—. In some embodiments, the cancer comprises cells that overexpress CD36. In some related aspects, the disclosure provides related medical uses and related uses of such compounds for the manufacture of a medicament for treating such cancers.

In a further aspect, the disclosure provides a method of treating cancer, comprising administering to a subject in need thereof a compound of the following formula:

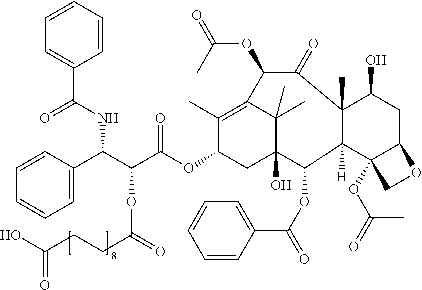

or a pharmaceutically acceptable salt thereof, wherein the cancer comprises cells that overexpress one or more fatty acid uptake proteins. In some embodiments, the cancer comprises cells that overexpress CD36. In some related aspects, the disclosure provides related medical uses and related uses of such compounds for the manufacture of a medicament for treating such cancers.

In a further aspect, the disclosure provides a method of treating cancer comprising administering to a subject in need thereof a compound of formula (I) in combination with one or more additional therapeutic agents:

A¹-X¹-X²-A² (I)

wherein: A¹ is a carboxylic acid group, a carboxylate anion, or a carboxylate ester; A² is a cytotoxic drug moiety, which has a molecular weight of no more than 1600 Da; X¹ is a hydrophobic group; and X² is a direct bond, an organic group, or a heteroatom group selected from the group consisting of —O—, —S—, —S(═O)—, —S(═O)₂—, —S—S—, —N═, ═N—, —N(H)—, —N═N—N(H)—, —N(H)—N═N—, —N(OH)—, or —N(═O)—; and wherein the cancer comprises cells that overexpress one or more fatty acid uptake proteins. In some embodiments, the cytotoxic drug moiety is a taxane moiety, such as a paclitaxel moiety. In some embodiments, the hydrophobic group is a C₁₂₋₂₂ hydrocarbylene group, which is optionally substituted. In some embodiments, X² is an organic group, such as a carbonyl group, i.e., —C(═O)—. In some embodiments, the cancer comprises cells that overexpress CD36. In some embodiments, the one or more additional therapeutic agents comprise one or more immunomodulating compounds. In some related aspects, the disclosure provides related medical uses and related uses of such compounds for the manufacture of a medicament for treating such cancers.

In a further aspect, the disclosure provides a method of treating cancer, comprising administering to a subject in need thereof a compound of the following formula in combination with one or more additional therapeutic agents:

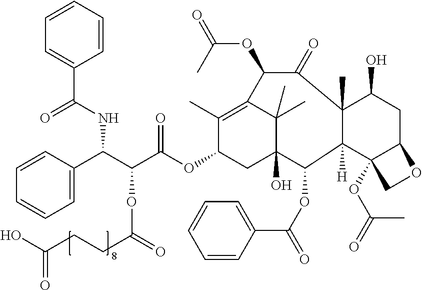

or a pharmaceutically acceptable salt thereof, wherein the cancer comprises cells that overexpress one or more fatty acid uptake proteins. In some embodiments, the cancer comprises cells that overexpress CD36. In some embodiments, the one or more additional therapeutic agents comprise one or more immunomodulating compounds. In some related aspects, the disclosure provides related medical uses and related uses of such compounds for the manufacture of a medicament for treating such cancers.

Further aspects and embodiments are provided in the drawings, the detailed description, the claims, and the abstract.

### BRIEF DESCRIPTION OF DRAWINGS

The following drawings are provided for purposes of illustrating various embodiments of the compounds, compositions, methods, and uses disclosed herein. The drawings are provided for illustrative purposes only, and are not intended to describe any preferred compounds or compositions or any preferred methods or uses, or to serve as a source of any limitations on the scope of the claimed inventions.

FIG. 1 shows non-limiting examples of compounds of formula (I), where the cytotoxic drug moiety is the paclitaxel moiety (PXT).

FIG. 2A and FIG. 2B show the change in cytotoxicity in HT-1080 cells as a function of CD36 inhibition.

FIG. 3 shows the change in cytotoxicity in HT-1080 cells as a function of CD36 inhibition.

FIG. 4 shows the change in cytotoxicity in MCF-7 cells as a function of CD36 inhibition.

FIG. 5 shows the change in cytotoxicity in HepG2 cells as a function of CD36 inhibition.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “hydrocarbon” refers to an organic group composed of carbon and hydrogen, which can be saturated or unsaturated, and can include aromatic groups. The term “hydrocarbyl” refers to a monovalent or polyvalent (e.g., divalent or higher) hydrocarbon moiety. In some cases, a divalent hydrocarbyl group is referred to as a “hydrocarbylene” group.

As used herein, “alkyl” refers to a straight or branched chain saturated hydrocarbon having 1 to 30 carbon atoms, which may be optionally substituted, as herein further described, with multiple degrees of substitution being allowed. Examples of “alkyl,” as used herein, include, but are not limited to, methyl, ethyl, n-propyl, isopropyl, isobutyl, n-butyl, sec-butyl, tert-butyl, isopentyl, n-pentyl, neopentyl, n-hexyl, and 2-ethylhexyl. In some instances, the “alkyl” group can be divalent, in which case, the group can alternatively be referred to as an “alkylene” group. Also, in some instances, one or more of the carbon atoms in the alkyl or alkylene group can be replaced by a heteroatom (e.g., selected from nitrogen, oxygen, or sulfur, including N-oxides, sulfur oxides, sulfur dioxides, and carbonyl groups, where feasible), and is referred to as a “heteroalkyl” or “heteroalkylene” group, respectively. Non-limiting examples include “oxyalkyl” or “oxyalkylene” groups, which refer to groups where a carbon atom in the alkyl or alkylene group is replaced by oxygen. Non-limiting examples of oxyalkyl or oxyalkylene groups include alkyl or alkylene chains that contain a carbonyl group, and also alkoxylates, polyalkylene oxides, and the like.

The number of carbon atoms in any group or compound can be represented by the terms. Thus, “C_{z}” refers to a group of compound having z carbon atoms, and “C_{x-y}”, refers to a group or compound containing from x to y, inclusive, carbon atoms. For example, “C₁₋₆ alkyl” represents an alkyl group having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl. The same logic applies to other types of functional groups, defined below.

As used herein, “alkenyl” refers to a straight or branched chain non-aromatic hydrocarbon having 2 to 30 carbon atoms and having one or more carbon-carbon double bonds, which may be optionally substituted, as herein further described, with multiple degrees of substitution being allowed. Examples of “alkenyl,” as used herein, include, but are not limited to, ethenyl, 2-propenyl, 2-butenyl, and 3-butenyl. In some instances, the “alkenyl” group can be divalent, in which case the group can alternatively be referred to as an “alkenylene” group. Also, in some instances, one or more of the carbon atoms in the alkenyl or alkenylene group can be replaced by a heteroatom (e.g., selected from nitrogen, oxygen, or sulfur, including N-oxides, sulfur oxides, sulfur dioxides, and carbonyl groups, where feasible), and is referred to as a “heteroalkenyl” or “heteroalkenylene” group, respectively.

As used herein, “cycloalkyl” refers to an aliphatic saturated or unsaturated hydrocarbon ring system having 3 to 20 carbon atoms, which may be optionally substituted, as herein further described, with multiple degrees of substitution being allowed. In some embodiments, the term refers only to saturated hydrocarbon ring systems, substituted as herein further described. Examples of “cycloalkyl,” as used herein, include, but are not limited to, cyclopropyl, cyclobutyl, cyclopentyl, cyclohexyl, cyclohexenyl, cycloheptyl, cyclooctyl, adamantyl, and the like. In some instances, the “cycloalkyl” group can be divalent, in which case the group can alternatively be referred to as a “cycloalkylene” group. Cycloalkyl and cycloalkylene groups can also be referred to herein as “carbocyclic rings.” Also, in some instances, one or more of the carbon atoms in the cycloalkyl or cycloalkylene group can be replaced by a heteroatom (e.g., selected independently from nitrogen, oxygen, silicon, or sulfur, including N-oxides, sulfur oxides, and sulfur dioxides, where feasible), and is referred to as a “heterocyclyl” or “heterocyclylene” group, respectively. The term “heterocyclic ring” can also be used interchangeably with either of these terms. In some embodiments, the cycloalkyl and heterocyclyl groups are fully saturated. In some other embodiments, the cycloalkyl and heterocyclyl groups can contain one or more carbon-carbon double bonds.

As used herein, “halogen,” “halogen atom,” or “halo” refer to a fluorine, chlorine, bromine, or iodine atom. In some embodiments, the terms refer to a fluorine or chlorine atom.

As used herein, the terms “organic group,” “organic moiety,” or “organic residue” refer to a monovalent or polyvalent functional group having at least one carbon atom, which optionally contains one or more additional atoms selected from the group consisting of hydrogen atoms, halogen atoms, nitrogen atoms, oxygen atoms, phosphorus atoms, and sulfur atoms, and which does not include covalently bound metal or semi-metal atoms. In some embodiments, these terms can include metal salts of organic groups, such as alkali metal or alkaline earth metal salts of organic anions.

As used herein, the term “pharmacophore” refers to a type of organic functional group. Standard pharmacophores are hydrophobic pharmacophores, hydrogen-bond donating pharmacophores, hydrogen-bond accepting pharmacophores, positive ionizable pharmacophores, and negative ionizable pharmacophores. The classification of organic functional groups within a compound is carried out according to standard classification systems known in the art.

As used herein, the terms “hydrophobic group,” “hydrophobic moiety,” or “hydrophobic residue” refer to an organic group that consists essentially of hydrophobic pharmacophores. In some embodiments, the terms refer to an organic group that consists of hydrophobic pharmacophores.

As used herein, the terms “hydrophilic group,” “hydrophilic moiety,” or “hydrophilic residue” refer to an organic group that comprises one pharmacophores selected from the group consisting of hydrogen bond donors, hydrogen bond acceptors, negative ionizable groups, or positive ionizable groups. In some embodiments, the terms refer to an organic group that consist essentially of pharmacophores selected from the group consisting of hydrogen bond donors, hydrogen bond acceptors, negative ionizable groups, or positive ionizable groups.

As used herein, the term “drug moiety” refers to a drug compound, or a pharmaceutically acceptable salt thereof, where an atom or a group of atoms is absent, thereby creating a monovalent or polyvalent moiety. In some embodiments, for example, a hydrogen atom is absent, thereby creating a monovalent moiety. In some other embodiments, a functional group, such as an —OH moiety, an —NH₂ moiety, or a —COOH, moiety is absent. In some embodiments, the drug moiety is a “cytotoxic drug moiety,” which refers to a drug moiety (as defined above) of a cytotoxic drug compound. In some further embodiments, the drug moiety is an “intracellularly active cytotoxic drug compound,” which refers to a cytotoxic drug moiety (defined above) whose primary cytotoxic effect occurs inside of the cell. For example, anti-folate compounds, such as gemcitabine, whose primary cytotoxic effect occurs outside of the cell (by blocking folate channels) is not intracellularly active cytotoxic drugs. One non-limiting example of a “drug moiety,” (in this case, a “paclitaxel moiety”) is the moiety of the following formula:

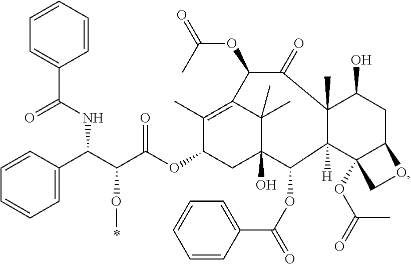 [SMILES: *O[C@@H](C(=O)O[C@H]1C[C@@]2(O)[C@@H](OC(=O)c3ccccc3)[C@@H]3[C@]4(OC(C)=O)CO[C@@H]4C[C@H](O)[C@@]3(C)C(=O)[C@H](OC(C)=O)C(=C1C)C2(C)C)[C@@H](NC(=O)c1ccccc1)c1ccccc1]

where a hydrogen atom is absent to create a monovalent moiety that, within a compound, bonds to the rest of the molecule through the remaining oxygen atom. Note that the term “drug moiety” is not limited to any particular procedure for making such compounds.

Various methods of drawing chemical structures are used herein. In some instances, the bond line-structure method is used to depict chemical compounds or moieties. In the line-structure method, the lines represent chemical bonds, and the carbon atoms are not explicitly shown (but are implied by the intersection of the lines). The hydrogen atoms are also not explicitly shown, except in instances where they are attached to heteroatoms. Heteroatoms, however, are explicitly shown. Thus, using that methodology, the structures shown below are for 2-methylpropane, 1-methoxypropane, and 1-propanol:

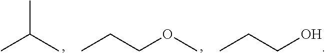

In that methodology, aromatic rings are typically represented merely by one of the contributing resonance structures. Thus, the following structures are for benzene, pyridine, and pyrrole:

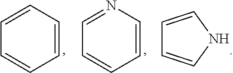

As used herein, a “protein binding moiety” is a moiety that binds non-covalently to one or more sites on a protein with a binding constant (K_{b}) of at least 100 M⁻¹ in water at 25° C.

As used herein, “amino acid” refers to a compound having the structure H₂N—R^{x}—COOH, where R^{x} is an organic group, and where the NH₂ may optionally combine with Rx (e.g., as in the case of proline). The term includes any known amino acids, including, but not limited to, alpha amino acids, beta amino acids, gamma amino acids, delta amino acids, and the like. In some embodiments, the term can refer to alpha amino acids.

As used herein, “hydroxy acid” refers to a compound having the structure HO—R^{y}—COOH, where R^{y} is an organic group. Non-limiting examples include glycolic acid, lactic acid, and caprolactone.

As used herein, “alkanol amine” refers to a compound having the structure HO—R^{z}—NH₂, where R^{z} is an optionally substituted alkylene group. Non-limiting examples include ethanol amine.

As used herein, “administer” or “administering” means to introduce, such as to introduce to a subject a compound or composition. The term is not limited to any specific mode of delivery, and can include, for example, subcutaneous delivery, intravenous delivery, intramuscular delivery, intracisternal delivery, delivery by infusion techniques, transdermal delivery, oral delivery, nasal delivery, and rectal delivery. Furthermore, depending on the mode of delivery, the administering can be carried out by various individuals, including, for example, a health-care professional (e.g., physician, nurse, etc.), a pharmacist, or the subject (i.e., self-administration).

As used herein, “treat” or “treating” or “treatment” can refer to one or more of:

delaying the progress of a disease, disorder, or condition; controlling a disease, disorder, or condition; ameliorating one or more symptoms characteristic of a disease, disorder, or condition; or delaying the recurrence of a disease, disorder, or condition, or characteristic symptoms thereof, depending on the nature of the disease, disorder, or condition and its characteristic symptoms. In the context of cancer, the terms “treat” or “treating” or “treatment” can, among other things, refer to inducing apoptosis of cancerous cells, reducing the size of a cancerous tumor, or inducing or enhancing an immune response against one or more cancerous cells, where the immune response has the effect of inducing apoptosis, reducing the size of a tumor, or the like.

As used herein, the term “in combination with,” such as when one compound is administered in combination with another compound, means that the two compounds are administered in a manner such that one or more biological effects of administering the first compound remain present when the second compound is administered. The two compounds need not be administered in a common dosage form or at substantially the same time. For example, in the context of cancer treatment, the two compounds could be administered one or several weeks apart from each other. For example, certain small-molecule cytotoxins (or prodrugs thereof) induce an immuno-priming, whereby the small-molecule cytotoxins (or prodrugs thereof) induce cell death in a manner that tends to improve the effectiveness of subsequent treatment using an immunomodulating agent. In such instances, the two compounds may be administered “in combination with each other,” even though initial administration of the small-molecule cytotoxin to the subject may precede administration of the immunomodulating agent to the subject by several weeks or several months.

As used herein, “subject” refers to any mammal such as, but not limited to, humans, horses, cows, sheep, pigs, mice, rats, dogs, cats, and primates such as chimpanzees, gorillas, and rhesus monkeys. In some embodiments, the “subject” is a human. In some such embodiments, the “subject” is a human who exhibits one or more symptoms characteristic of a disease, disorder, or condition. The term “subject” does not require one to have any particular status with respect to a hospital, clinic, or research facility (e.g., as an admitted patient, a study participant, or the like).

As used herein, the term “compound” includes free acids, free bases, and salts thereof.

As used herein, the term “pharmaceutical composition” is used to denote a composition that may be administered to a mammalian host, e.g., orally, topically, parenterally, by inhalation spray, or rectally, in unit dosage formulations containing conventional non-toxic carriers, diluents, adjuvants, vehicles and the like. The term “parenteral” as used herein, includes subcutaneous injections, intravenous, intramuscular, intracisternal injection, or by infusion techniques.

Also included within the scope of the disclosure are the individual enantiomers of the compounds represented by Formula (I) or pharmaceutically acceptable salts thereof, as well as any wholly or partially racemic mixtures thereof. The disclosure also covers the individual enantiomers of the compounds represented by Formula (I) or pharmaceutically acceptable salts thereof, as well as mixtures with diastereoisomers thereof in which one or more stereocenters are inverted. Unless otherwise stated, structures depicted herein are also meant to include compounds which differ only in the presence of one or more isotopically enriched atoms. For example, compounds having the present structure, except for the replacement of a hydrogen atom by a deuterium or tritium, or the replacement of a carbon atom by a ¹³C- or ¹⁴C-enriched carbon are within the scope of the disclosure.

As used herein, “mix” or “mixed” or “mixture” refers broadly to any combining of two or more compositions. The two or more compositions need not have the same physical state; thus, solids can be “mixed” with liquids, e.g., to form a slurry, suspension, or solution. Further, these terms do not require any degree of homogeneity or uniformity of composition. This, such “mixtures” can be homogeneous or heterogeneous, or can be uniform or non-uniform. Further, the terms do not require the use of any particular equipment to carry out the mixing, such as an industrial mixer.

As used herein, “optionally” means that the subsequently described event(s) may or may not occur. In some embodiments, the optional event does not occur. In some other embodiments, the optional event does occur one or more times.

As used herein, “substituted” refers to substitution of one or more hydrogen atoms of the designated moiety with the named substituent or substituents, multiple degrees of substitution being allowed unless otherwise stated, provided that the substitution results in a stable or chemically feasible compound. A stable compound or chemically feasible compound is one in which the chemical structure is not substantially altered when kept at a temperature from about −80° C. to about +40° C., in the absence of moisture or other chemically reactive conditions, for at least a week. As used herein, the phrases “substituted with one or more . . . ” or “substituted one or more times . . . ” refer to a number of substituents that equals from one to the maximum number of substituents possible based on the number of available bonding sites, provided that the above conditions of stability and chemical feasibility are met.

Exemplary optional substituents include, among others, a halogen, particularly Cl or F, an oxo, a hydroxyl group, an unsubstituted alkyl group, a halogen-substituted alkyl group, an unsubstituted alkoxy group, an unsubstituted —S-alkyl group, an amino group (—NH₂), a mono- or dialkyl amino group, an alkoxy-substituted alkyl group, or a —S-alkyl substituted alkyl group. Alkyl and alkoxy groups of such substituents may include 1-6 carbon atoms or 1-3 carbon atoms.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present. As used herein, the phrases “consist essentially of,” “consists essentially of,” and “consisting essentially of” refer to groups that are open, but which only includes additional unnamed members that would not materially affect the basic characteristics of the claimed subject matter.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, the various functional groups represented will be understood to have a point of attachment at the functional group having the hyphen or dash (-) or a dash used in combination with an asterisk (*). In other words, in the case of —CH₂CH₂CH₃ or *—CH₂CH₂CH₃, it will be understood that the point of attachment is the CH₂ group at the far left. If a group is recited without an asterisk or a dash, then the attachment point is indicated by the plain and ordinary meaning of the recited group.

As used herein, multi-atom bivalent species are to be read from left to right. For example, if the specification or claims recite A-D-E and D is defined —OC(O)—, the resulting group with D replaced is: A-OC(O)-E and not A-C(O)O-E.

Other terms are defined in other portions of this description, even though not included in this subsection.

### Methods of Using Modified Cytotoxins and Related Medical Uses

In at least one aspect, the disclosure provides methods of treating cancer, comprising administering a compound of formula (I) to a subject in need thereof:

A¹-X¹-X²-A² (I)

wherein: A¹ is a carboxylic acid group, a carboxylate anion, or a carboxylate ester; A² is a cytotoxic drug moiety; X¹ is a hydrophobic group; and X² is a direct bond, an organic group, or a group selected from the group consisting of —O—, —S—, —S(═O)—, —S(═O)₂—, —S—S—, —N═, ═N—, —N(H)—, —N═N—N(H)—, —N(H)—N═N—, —N(OH)—, or —N(═O)—; and wherein the cancer comprises cells that overexpress one or more fatty acid uptake proteins.

In a related aspect, the disclosure provides uses of a compound of formula (I) to treat cancer:

A¹-X¹-X²-A² (I)

wherein: A¹ is a carboxylic acid group, a carboxylate anion, or a carboxylate ester; A² is a cytotoxic drug moiety; X¹ is a hydrophobic group; and X² is a direct bond, an organic group, or a group selected from the group consisting of —O—, —S—, —S(═O)—, —S(═O)₂—, —S—S—, —N═, ═N—, —N(H)—, —N═N—N(H)—, —N(H)—N═N—, —N(OH)—, or —N(═O)—; and wherein the cancer comprises cells that overexpress one or more fatty acid uptake proteins.

In another related aspect, the disclosure provides uses of a compound of formula (I) in the manufacture of a medicament for the treatment of cancer:

A¹-X¹-X²-A² (I)

wherein: A¹ is a carboxylic acid group, a carboxylate anion, or a carboxylate ester; A² is a cytotoxic drug moiety; X¹ is a hydrophobic group; and X² is a direct bond, an organic group, or a group selected from the group consisting of —O—, —S—, —S(═O)—, —S(═O)₂—, —S—S—, —N═, ═N—, —N(H)—, —N═N—N(H)—, —N(H)—N═N—, —N(OH)—, or —N(═O)—; and wherein the cancer comprises cells that overexpress one or more fatty acid uptake proteins.

In some embodiments of any of the foregoing related aspects and embodiments, A¹ is selected from the group consisting of a carboxylic acid group (—COOH), a carboxylate anion (—COO⁻), or a carboxylate ester (e.g., —COOR^{a}, where R^{a} is an organic group such as an alkyl or alkoxylate group). In some such embodiments, A¹ is a carboxylic acid group. In some such embodiments, A¹ is a carboxylate ester group.

In some embodiments of any of the foregoing related aspects and embodiments, X¹ is C₈₋₃₀ hydrocarbylene, which is optionally substituted. In some further embodiments, X¹ is C₁₂₋₂₂ hydrocarbylene, which is optionally substituted. In some further embodiments, X¹ is C₁₂₋₂₂ alkylene. In some further embodiments, X¹ is —(CH₂)₁₂—, —(CH₂)₁₄—, —(CH₂)₁₆—, —(CH₂)₁₈—, —(CH₂)₂₀—, or —(CH₂)₂₂—. In some other embodiments, X¹ is —(CH₂)₁₆—. In some further embodiments, X¹ is C₁₂₋₂₂ alkenylene. In some further such embodiments, X¹ is —(CH₂)₇—CH═CH—(CH₂)₇—.

In some further embodiments of any of the foregoing related aspects or embodiments, X¹ is C₁₂₋₂₂ hydrocarbylene, which is optionally substituted. In some such embodiments, X¹ is C₁₂₋₂₂ hydrocarbylene. In some further such embodiments, X¹ is C₁₄₋₂₂ hydrocarbylene. In some further such embodiments, X¹ is C₁₆₋₂₂ hydrocarbylene. In some embodiments of any of the aforementioned embodiments, X¹ is C₁₂₋₂₂ hydrocarbylene, wherein A¹ and X² (or, if X² is a direct bond, A²) are separated from each other by at least 6, or by at least 8, or by at least 10, or by at least 12, or by at least 14, carbon atoms. In some further such embodiments, X¹ is C₁₄₋₂₂ hydrocarbylene, wherein A¹ and X² (or, if X² is a direct bond, A²) are separated from each other by at least 6, or by at least 8, or by at least 10, or by at least 12, or by at least 14, carbon atoms. In some further such embodiments, X¹ is C₁₆₋₂₂ hydrocarbylene, wherein A¹ and X² (or, if X² is a direct bond, A²) are separated from each other by at least 6, or by at least 8, or by at least 10, or by at least 12, or by at least 14, carbon atoms. In some further embodiments of any of the aforementioned embodiments, X¹ is C₁₂₋₂₂ straight-chain alkylene, or C₁₄₋₂₂ straight-chain alkylene, or C₁₆₋₂₂ straight-chain alkylene. In some further embodiments of any of the aforementioned embodiments, X¹ is C₁₂₋₂₂ straight-chain alkenylene, or C₁₄₋₂₂ straight-chain alkenylene, or C₁₆₋₂₂ straight-chain alkenylene.

In some embodiments of any of the foregoing related aspects and embodiments, X² is a direct bond. In some other embodiments of any of the foregoing related aspects and embodiments, X² is an organic group. In some embodiments, X² is a hydrophilic group. In some embodiments, X² is a heteroalkylene group.

In any of the aforementioned embodiments where X² is an organic group, X² can contain any suitable number of carbon atoms. In some embodiments, for example, X² contains from 1 to 100 carbon atoms, or from 1 to 50 carbon atoms, or from 1 to 25 carbon atoms, or from 1 to 10 carbon atoms, or from 1 to 6 carbon atoms.

In any of the aforementioned embodiments where X² is a heteroalkylene group, X² can contain any suitable number of carbon atoms. In some embodiments, for example, X² contains from 1 to 100 carbon atoms, or from 1 to 50 carbon atoms, or from 1 to 25 carbon atoms, or from 1 to 10 carbon atoms, or from 1 to 6 carbon atoms.

In some of the aforementioned embodiments, X² can contain certain groups. Some non-limiting examples of such groups that X² can contain are polyalkylene oxide groups, such as polyethylene glycol (PEG) and various polypeptide chains.

In some embodiments, X² is an organic group selected from the group consisting of —C(═O)—, —C≡C—, —C(H)═C(H)—, —C(═O)—O—, —O—C(═O)—, —C(═O)—NH—, —NH—C(═O)—, —NH—C(═O)—O—, —O—(C═O)—NH—, —O—C(═O)—O—, —C(═N—NH₂)—, —C(═N—R^{b})—(where R^{b} is a hydrogen atom or an alkyl group), —C(═N—OH)—, —NH—C(═O)—NH—, —NH—C(═S)—NH—, —NH—C(═S)—O—, —O—C(═S)—NH—, —NH—C(═O)—S—, —S—C(═O)—NH—, —NH—C(═S)—S—, —S—C(═S)—NH—, and the cyclic structures shown below:

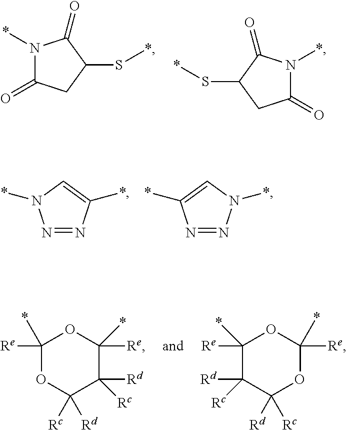

where R^{c}, R^{d}, and R^{e} are, independently at each occurrence, a hydrogen atom or C₁₋₁₀ alkyl. In some further embodiments, X² is —C(═O)—.

In some embodiments, X² is a group selected from the group consisting of —O—, —S—, —S(═O)—, —S(═O)₂—, —S—S—, —N═, ═N—, —N(H)—, —N═N—N(H)—, —N(H)—N═N—, —N(OH)—, and —N(O)—.

In some embodiments, X² comprises one or more moieties selected from the group consisting of: —C(═O)—, —O—C(═O)—, —NH—C(═O)—, one or more moieties formed from a alkylene glycols, one or more units formed from alkanol amines, one or more units formed from amino acids, and one or more units formed from hydroxyl acids. Thus, in some embodiments, X² comprises one or more moieties formed from alkylene glycols, such as a short poly(ethylene glycol) chain having 1 to 25 ethylene glycol units. In some embodiments, X² comprises one or more moieties formed from amino acids, such as an oligopeptide chain having 1 to 25 amino acid units. In some embodiments, X² comprises one or more moieties formed from hydroxy acids, such as moieties formed from glycolic acid, lactic acid, or caprolactone. In some embodiments, X² comprises a combination of a poly(ethylene glycol) chain having 1 to 25 ethylene glycol units and an oligopeptide having 1 to 25 amino acid units, and optionally one or more units formed from hydroxy acids.

In any of the above embodiments, the selection of X² will depend on the type of functional group through which it is linked to the cytotoxic drug moiety, so as to avoid making compounds that are chemically unstable or impossible. The skilled artisan will be able to select combinations of X² and A² that result in chemically stable compounds, which are compounds in which the chemical structure is not substantially altered when kept at a temperature from about −80° C. to about +40° C., in the absence of moisture or other chemically reactive conditions, for at least a week.

In some embodiments of any of the foregoing related aspects and embodiments, A² can be any suitable cytotoxic drug moiety. In some embodiments, the cytotoxic drug moiety is a small-molecule drug moiety, such as a cytotoxic drug moiety having a molecular weight of or no more than 1600 Da, or no more than 1500 Da, or no more than 1400 Da, or no more than 1300 Da, no more than 1200 Da, or no more than 1100 Da, or no more than 1000 Da, or no more than 900 Da. Such drug moieties can be organic moieties, or can also be moieties that contain inorganic atoms (e.g., platinum). In some embodiments, however, the cytotoxic drug moiety is an organic moiety. In some embodiments, the cytotoxic drug moiety is an intracellularly active cytotoxic drug moiety. In some embodiments, the cytotoxic drug moiety is a taxane moiety (such as a paclitaxel moiety), a topoisomerase I inhibitor moiety (such as an etoposide moiety), a topoisomerase II inhibitor moiety (such as a topotecan moiety), an alkylating agent moiety (such as a cyclophosphamide moiety), an anthracycline moiety (such as a doxorubicin moiety), a purine analog moiety (such as a fludarabine moiety), a vinca alkaloid moiety (such as a vincristine moiety), a kinase inhibitor moiety (such as an imatinib moiety), a ubiquitin ligase modulator moiety (such as a lenalidomide moiety), an androgen receptor agonist moiety (such as an abiraterone moiety), a proteasome inhibitor (such as a bortezomib inhibitor), a Hedgehog signaling pathway modulator moiety (such as a vismodegib moiety), or an epothilone moiety (such as an ixabepilone moiety). In some embodiments, the cytotoxic drug moiety is a taxane moiety.

In some embodiments of any of the aforementioned embodiments, the cytotoxic drug moiety is a moiety selected from the group consisting of: a paclitaxel moiety, an etoposide moiety, a cyclophosphamide moiety, a chlorambucil moiety, a doxorubicin moiety, a daunorubicin moiety, a dactinomycin moiety, an amifostine moiety, a fludarabine moiety, a topotecan moiety, an ifosfamide moiety, a vincristine moiety, a carboplatin moiety, a vinblastine moiety, an imatinib moiety, a lenalidomide moiety, an abiraterone moiety, an erlotinib moiety, a bortezomib moiety, an oxaliplatin moiety, a methotrexate moiety, a carfilzomib moiety, a crizotinib moiety, a vismodegib moiety, a ponatinib moiety, a tivozanib moiety, a carbozantinib moiety, an epirubicin moiety, a docetaxel moiety, a cisplatin moiety, an eribulin moiety, an ixabepilone moiety, a vinorelbine moiety, an everolimus moiety, a mytomycin C moiety, a sunitinib moiety, an irinotecan moiety, a leicovorim moiety, a tretinoin moiety, an allopurinol moiety, an asparaginase moiety, a bendamustine moiety, a bleomycin moiety, a cytarabine moiety, a dacarbazine moiety, a filgrastim moiety, a hydroxycarbamide moiety, a mercaptopurine moiety, a mesna moiety, a procarbazine moiety, a thioguanine moiety, and pharmaceutically acceptable salts of any of the foregoing. In some further such embodiments, the cytotoxic drug moiety is a moiety selected from the group consisting of: a paclitaxel moiety, an etoposide moiety, a gemcitabine moiety, a cyclophosphamide moiety, a chlorambucil moiety, a doxorubicin moiety, a daunorubicin moiety, a 5-fluorouracil moiety, a dactinomycin moiety, an amifostine moiety, a fludarabine moiety, a topotecan moiety, an ifosfamide moiety, a vincristine moiety, a vinblastine moiety, an imatinib moiety, a lenalidomide moiety, a pemetrexed moiety, an abiraterone moiety, an erlotinib moiety, a bortezomib moiety, a methotrexate moiety, a carfilzomib moiety, a crizotinib moiety, a vismodegib moiety, a ponatinib moiety, a tivozanib moiety, a carbozantinib moiety, an epirubicin moiety, a docetaxel moiety, an eribulun moiety, an ixabepilone moiety, a vinorelbine moiety, an everolimus moiety, a mytomycin C moiety, a sunitinib moiety, an irinotecan moiety, a leicovorim moiety, and pharmaceutically acceptable salts of any of the foregoing. In some further such embodiments, the cytotoxic drug moiety is selected from the group consisting of: a paclitaxel moiety, a gemcitabine moiety, a doxorubicin moiety, a 5-fluorouracil moiety, a methotrexate moiety, and a pemetrexed moiety. In some further such embodiments, the cytotoxic drug moiety is a paclitaxel moiety. In some further such embodiments, the cytotoxic drug moiety is a gemcitabine moiety. In some further such embodiments, the cytotoxic drug moiety is a 5-fluorouracil moiety. In some further such embodiments, the cytotoxic drug moiety is a pemetrexed moiety.

In the aforementioned embodiments, the named moieties can have any suitable chemical form. In some embodiments of any of the aforementioned embodiments, the cytotoxic drug moieties are moieties where a hydrogen atom is absent from the named drug compound, or a pharmaceutically acceptable salt thereof. As a non-limiting example, such a “paclitaxel moiety” would include the moiety of the following formula:

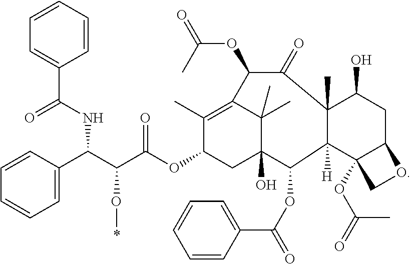 [SMILES: *O[C@@H](C(=O)O[C@H]1C[C@@]2(O)[C@@H](OC(=O)c3ccccc3)[C@@H]3[C@]4(OC(C)=O)CO[C@@H]4C[C@H](O)[C@@]3(C)C(=O)[C@H](OC(C)=O)C(=C1C)C2(C)C)[C@@H](NC(=O)c1ccccc1)c1ccccc1]

In any of the foregoing embodiments, -X²-X¹-A¹ can have any suitable structure, so long as those combinations result in stable chemical structures that would be suitable for pharmaceutical use. In some such embodiments, -X²-X¹-A¹ is —C(═O)—(CH₂)₁₀—CH₃, —C(═O)—(CH₂)₁₂—CH₃, —C(═O)—(CH₂)₁₄—CH₃, or —C(═O)—(CH₂)₁₆—CH₃. In some other such embodiments, -X²-X¹-A¹ is —C(═O)—(CH₂)₁₀—C(═O)—OH, —C(═O)—(CH₂)₁₂—C(═O)—OH, —C(═O)—(CH₂)₁₄—C(═O)—OH, or —C(═O)—(CH₂)₁₆—C(═O)—OH.

The selection of -X²-X¹-A¹ can depend on the nature of the connection to the drug moiety. For example, in embodiments where the -X²-X¹-A¹ connects to an oxygen atom or an NH group on the drug moiety, as is the case for entries HA1, HA2, HA9, HA12, HA14, HA15, HA16, HA19, HA20, HA21, HA22, HA23, and HA24 in Table 1, then -X²-X¹⁻-A¹ is selected from the group consisting of: —C(═O)—(CH₂)ₙ₁—C(═O)—OH; —C(═O)—(CH₂)ₙ₁—C(═O)—OCH₃; —C(═O)—(CH₂)ₙ₁—CH₃; —C(═O)-(C₁₋₆ alkylene)-C(═O)—O—(CH₂)ₙ₂—C(═O)—OH; —C(═O)-(C₁₋₆ alkylene)-NH—C(═O)—(CH₂)ₙ₁—C(═O)—OH; —C(═O)-(C₁₋₆ alkylene)-C(═O)—O—[(CH₂)₂—O—]ₙ₃(CH₂)ₙ₂—C(═O)—OH; —C(═O)—O—(CH₂)ₙ₂—C(═O)—OH; and —C(═O)—NH—(CH₂)ₙ₂—C(═O)—OH; wherein n1 is an integer 12 to 24, n2 is an integer from 13 to 25, and n3 is an integer from 1 to 25. In some further such embodiments, -X²-X¹-A¹ is selected from the group consisting of: —C(═O)—(CH₂)ₙ₁—C(═O)—OH; —C(═O)—(CH₂)ₙ₁—C(═O)—OCH₃; —C(═O)-(C₁₋₆ alkylene)-C(═O)—O—(CH₂)ₙ₂—C(═O)—OH; —C(═O)-(C₁₋₆ alkylene)-NH—C(═O)—(CH₂)ₙ₁—C(═O)—OH; —C(═O)-(C₁₋₆ alkylene)-C(═O)—O—[(CH₂)₂—O—]ₙ₃(CH₂)ₙ₂—C(═O)—OH; —C(═O)—O—(CH₂)ₙ₂—C(═O)—OH; and —C(═O)—NH—(CH₂)ₙ₂—C(═O)—OH. In some further such embodiments, -X²-X¹-A¹ is selected from the group consisting of: —C(═O)—(CH₂)ₙ₁—C(═O)—OH; —C(═O)—O—(CH₂)ₙ₂—C(═O)—OH; and —C(═O)—NH—(CH₂)ₙ₂—C(═O)—OH. In some other embodiments, -X²-X¹-A¹ is —C(═O)-(C₁₋₆ alkylene)-O—C(═O)—(CH₂)ₙ₁—C(═O)—OH, where n1 is an integer from 12 to 24. In some embodiments of any of the aforementioned embodiments, n1 is an integer from 14 to 22, or from 16 to 20. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, -X²-X¹-A¹ is —C(═O)-(C₁₋₆ alkylene)-C(═O)—O—(CH₂)ₙ₃—OH, where n3 is an integer from 14 to 26, or an integer from 16 to 24, or an integer from 18 to 22.

In embodiments where the -X²-X¹-A¹ connects to an >N group on the drug moiety, as is the case for entries HA3 and HA4 in Table 1, then -X²-X¹-A¹ is selected from the group consisting of: —CH₂—O—C(═O)—(CH₂)ₙ₁—C(═O)—OH; —CH₂—O—C(═O)—(CH₂)ₙ₁—C(═O)—OCH₃; —CH₂—O—C(═O)—(CH₂)ₙ₁—-CH₃; —CH₂—O—C(═O)-(C₁₋₆ alkylene)-C(═O)—O—(CH₂)ₙ₂—C(50 O)—OH; —CH₂—O—C(═O)-(C₁₋₆ alkylene)-NH—C(═O)—(CH₂)ₙ₁—C(═O)—OH; —CH₂—O—C(═O)-(C₁₋₆ alkylene)-C(═O)—O—[(CH₂)₂—O—]ₙ₃(CH₂)ₙ₂—C(═O)—OH; —CH₂—O—C(═O)—O—(CH₂)ₙ₂—C(═O)—OH; and —CH₂—O—C(═O)—NH—(CH₂)ₙ₂—C(═O)—OH; wherein n1 is an integer 12 to 24, n2 is an integer from 13 to 25, and n3 is an integer from 1 to 25. In some further such embodiments, -X²-X¹-A¹ is selected from the group consisting of: —CH₂—O—C(═O)—(CH₂)ₙ₁—C(═O)—OH; —CH₂—O—C(═O)—(CH₂)ₙ₁—C(═O)—OCH₃; —CH₂—O—C(═O)-(C₁₋₆ alkylene)-C(═O)—O—(CH₂)ₙ₂—C(═O)—OH; —CH₂—O—C(═O)-(C₁₋₆ alkylene)-NH—C(═O)—(CH₂)ₙ₁—C(═O)—OH; —CH₂—O—C(═O)-(C₁₋₆ alkylene)-C(═O)—O—[(CH₂)₂—O—]ₙ₃(CH₂)ₙ₂—C(═O)—OH; —CH₂—O—C(═O)—O—(CH₂)ₙ₂—C(═O)—OH; and —C(═O)—NH—(CH₂)ₙ₂—C(═O)—OH. In some further such embodiments, -X²-X¹-A¹ is selected from the group consisting of: —CH₂—O—C(═O)—(CH₂)ₙ₁—C(═O)—OH; —CH₂—O—C(═O)—O—(CH₂)ₙ₂—C(═O)—OH; and —CH₂—O—C(═O)—NH—(CH₂)ₙ₂—C(═O)—OH. In some embodiments of any of the aforementioned embodiments, n1 is an integer from 14 to 22, or from 16 to 20. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, -X²-X¹-A¹ is —CH₂—O—C(═O)-(C₁₋₆ alkylene)-C(═O)—O—(CH₂)ₙ₃—OH, where n3 is an integer from 14 to 26, or an integer from 16 to 24, or an integer from 18 to 22.

In embodiments where the -X²-X¹-A¹ connects to a —C(═O) group on the drug moiety, as is the case for entries HA5, HA6, HA7, HA8, HA11, and HA17 in Table 1, then -X²-X¹-A¹ is selected from the group consisting of: —O—(CH₂)ₙ₂—C(═O)—OH; —NH—(CH₂)ₙ₂—C(═O)—OH; —NH-(C₁₋₆ alkylene)-O—C(═O)—(CH₂)ₙ₁—C(═O)—OH; —O-(C₁₋₆ alkylene)-O—C(═O)—(CH₂)ₙ₁—C(═O)—OH; —NH-(C₁₋₆ alkylene)-O—C(═O)—(CH₂)ₙ₁—C(═O)—OCH₃; —O-(C₁₋₆ alkylene)-O—C(═O)—(CH₂)ₙ₁—C(═O)—OCH₃; —NH-(C₁₋₆ alkylene)-O—C(═O)—(CH₂)ₙ₁—CH₃; —O-(C₁₋₆ alkylene)-O—C(═O)—(CH₂)ₙ₁—CH₃; —NH-(C₁₋₆ alkylene)-C(═O)—O—[(CH₂)₂—O—]ₙ₃(CH₂)ₙ₂—C(═O)—OH; and —O-(C₁₋₆ alkylene)-C(═O)—O—[(CH₂)₂—O—]ₙ₃(CH₂)ₙ₂—C(═O)—OH; wherein n1 is an integer 12 to 24, n2 is an integer from 13 to 25, and n3 is an integer from 1 to 25. In some further such embodiments, -X²-X¹-A¹ is selected from the group consisting of: —O—(CH₂)ₙ₂—C(═O)—OH; —NH—(CH₂)ₙ₂—C(═O)—OH; —NH-(C₁₋₆ alkylene)-O—C(═O)—(CH₂)ₙ₁—C(═O)—OH; —O-(C₁₋₆ alkylene)-O—C(═O)—(CH₂)ₙ₁—C(═O)—OH; —NH-(C₁₋₆ alkylene)-O—C(═O)—(CH₂)ₙ₁—C(═O)—OCH₃; and —O-(C₁₋₆ alkylene)-O—C(═O)—(CH₂)ₙ₁—C(═O)—OCH₃. In some further such embodiments, -X²-X¹⁻-A¹ is selected from the group consisting of: —O—(CH₂)ₙ₂—C(═O)—OH; —NH—(CH₂)ₙ₂—C(═O)—OH; —NH-(C₁₋₆ alkylene)-O—C(═O)—(CH₂)ₙ₁—C(═O)—OH; and —O-(C₁₋₆ alkylene)-O—C(═O)—(CH₂)ₙ₁—C(═O)—OH. In some embodiments of any of the aforementioned embodiments, n1 is an integer from 14 to 22, or from 16 to 20. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, -X²-X¹-A¹ is —O—(CH₂)ₙ₃—OH, where n3 is an integer from 14 to 26, or an integer from 16 to 24, or an integer from 18 to 22.

In embodiments where the -X²-X¹-A¹ connects to a C═* group on the drug moiety, as is the case for entries HA10, HA13, and HA18 in Table 1, then -X²-X¹-A¹ is selected from the group consisting of: ═N—O—(CH₂)ₙ₂—C(═O)—OH; ═N—NH—(CH₂)ₙ₂—C(═O)—OH; ═N—O—(CH₂)ₙ₂—C(═O)—OCH₃; ═N—NH—(CH₂)ₙ₂—C(═O)—OCH₃; ═N—O—(CH₂)ₙ₂—CH₃; ═N—NH—(CH₂)ₙ₂—CH₃; ═N—O—[(CH₂)₂—O—]ₙ₃(CH₂)ₙ₂—C(═O)—OH; and ═N—NH—[(CH₂)₂—O—]ₙ₃(CH₂)ₙ₂—C(═O)—OH; n2 is an integer from 13 to 25, and n3 is an integer from 1 to 25. In some further such embodiments, -X²-X¹-A¹ is selected from the group consisting of: ═N—O—(CH₂)ₙ₂—C(═O)—OH; ═N—NH—(CH₂)ₙ₂—C(═O)—OH; ═N—O—(CH₂)ₙ₂—C(═O)—OCH₃; and ═N—NH—(CH₂)ₙ₂—C(═O)—OCH₃. In some further such embodiments, -X²-X¹-A¹ is selected from the group consisting of: ═N—O—(CH₂)ₙ₂—C(═O)—OH and ═N—NH—(CH₂)ₙ₂—C(═O)—OH. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, -X²-X¹-A¹ is selected from the group consisting of: ═NO—(CH₂)ₙ₃—OH and ═N—NH—(CH₂)ₙ₃—OH, where n3 is an integer from 14 to 26, or an integer from 16 to 24, or an integer from 18 to 22.

The compounds described in any of the above embodiments can also exist as pharmaceutically acceptable salts. The term “pharmaceutically acceptable salts” refers to salts of the compounds which are not biologically or otherwise undesirable and are generally prepared by reacting the free base with a suitable organic or inorganic acid or by reacting the acid with a suitable organic or inorganic base. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium, and valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, there can be formed an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethanesulfonate, picrate, and the like.

The compounds above can be made by standard organic synthetic methods, such as those illustrated in: Wuts et al., Greene's Protective Groups in Organic Synthesis (4th ed., 2006); Larock, Comprehensive Organic Transformations (2nd ed., 1999); and Smith et al., March's Advanced Organic Chemistry: Reactions, Mechanisms, and Structure (6th ed., 2007). Specific non-limiting examples are shown below in the Examples.

The compounds of the foregoing embodiments, including their pharmaceutically acceptable salts, are useful as cytotoxic agents or prodrugs thereof, and are therefore useful as compounds for the treatment of cancer.

Table 3 (below) shows various examples of compounds that are contemplated by the present disclosure. Table 3 refers to various combinations of an A²-moiety with a -X²-X¹-A¹, which together form compounds of the present disclosure. Table 1 shows illustrative example moieties for the A²-moiety, wherein A² can be the moiety shown or can also be a pharmaceutically acceptable salt thereof. Table 2 shows illustrative example moieties for -X²-X¹-A¹. Table 3 shows non-limiting illustrative combinations of the moieties from Tables 1 and 2, which can come together to form compounds of the present disclosure.

The compounds disclosed in Table 3 can be made by methods analogous to those illustrated in the Examples, and by common synthetic methods known to those of ordinary skill in the art. Suitable methods of making such compounds are illustrated in: Wuts et al., Greene's Protective Groups in Organic Synthesis (4th ed., 2006); Larock, Comprehensive Organic Transformations (2nd ed., 1999); and Smith et al., March's Advanced Organic Chemistry: Reactions, Mechanisms, and Structure (6th ed., 2007).

TABLE 1
A²-Moieties

| | |
|---|---|
| 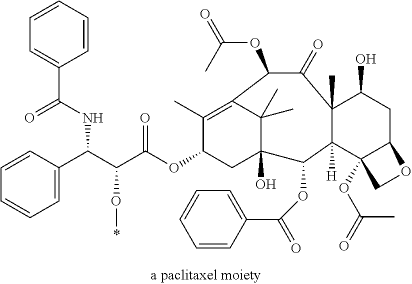 [SMILES: *O[C@@H](C(=O)O[C@H]1C[C@@]2(O)[C@@H](OC(=O)c3ccccc3)[C@@H]3[C@]4(OC(C)=O)CO[C@@H]4C[C@H](O)[C@@]3(C)C(=O)[C@H](OC(C)=O)C(=C1C)C2(C)C)[C@@H](NC(=O)c1ccccc1)c1ccccc1] | HA1 |
| 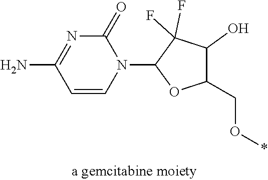 [SMILES: *OCC1OC(n2ccc(N)nc2=O)C(F)(F)C1O] | HA2 |
| 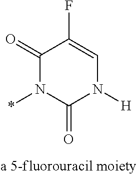 [SMILES: *n1c(=O)[nH]cc(F)c1=O] | HA3 |
| 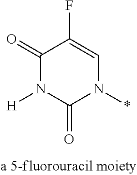 [SMILES: *n1cc(F)c(=O)[nH]c1=O] | HA4 |
| 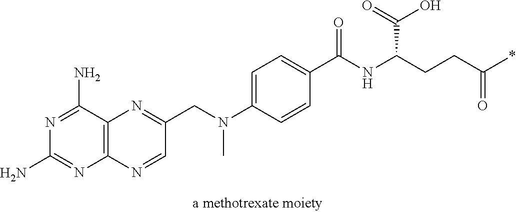 [SMILES: *C(=O)CC[C@H](NC(=O)c1ccc(N(C)Cc2cnc3nc(N)nc(N)c3n2)cc1)C(=O)O] | HA5 |
| 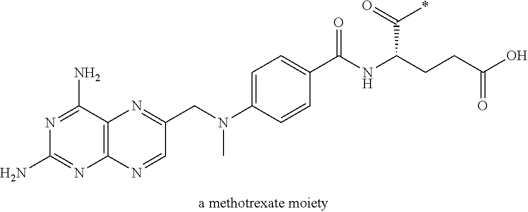 [SMILES: *C(=O)[C@H](CCC(=O)O)NC(=O)c1ccc(N(C)Cc2cnc3nc(N)nc(N)c3n2)cc1] | HA6 |
| 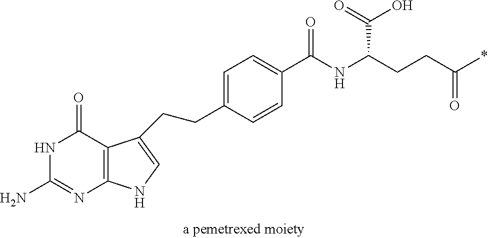 [SMILES: *C(=O)CC[C@H](NC(=O)c1ccc(CCc2c[nH]c3nc(N)[nH]c(=O)c23)cc1)C(=O)O] | HA7 |
| 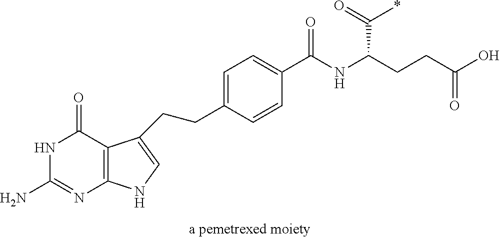 [SMILES: *C(=O)[C@H](CCC(=O)O)NC(=O)c1ccc(CCc2c[nH]c3nc(N)[nH]c(=O)c23)cc1] | HA8 |
| 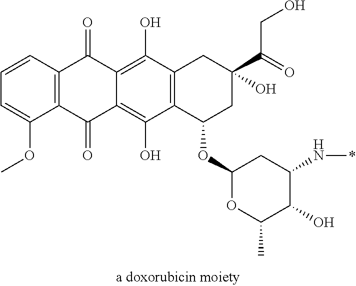 [SMILES: *N[C@H]1C[C@H](O[C@H]2C[C@](O)(C(=O)CO)Cc3c(O)c4c(c(O)c32)C(=O)c2c(OC)cccc2C4=O)O[C@@H](C)[C@H]1O] | HA9 |
| 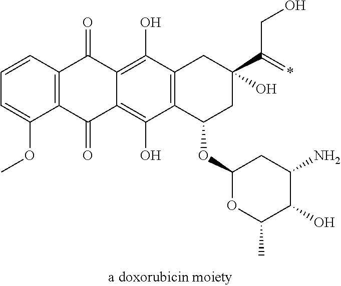 [SMILES: *=C(CO)[C@]1(O)Cc2c(O)c3c(c(O)c2[C@@H](O[C@H]2C[C@H](N)[C@H](O)[C@H](C)O2)C1)C(=O)c1c(OC)cccc1C3=O] | HA10 |
| 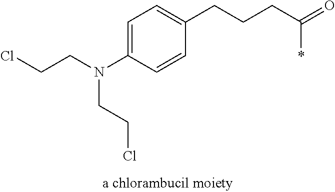 [SMILES: *C(=O)CCCc1ccc(N(CCCl)CCCl)cc1] | HA11 |
| 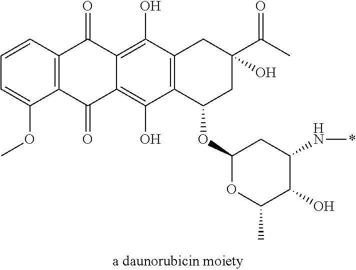 [SMILES: *N[C@H]1C[C@H](O[C@H]2C[C@](O)(C(C)=O)Cc3c(O)c4c(c(O)c32)C(=O)c2c(OC)cccc2C4=O)O[C@@H](C)[C@H]1O] | HA12 |
| 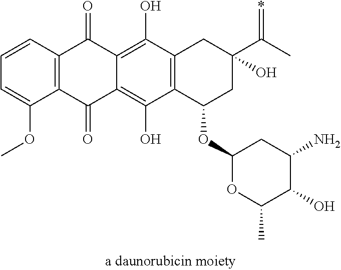 [SMILES: *=C(C)[C@]1(O)Cc2c(O)c3c(c(O)c2[C@@H](O[C@H]2C[C@H](N)[C@H](O)[C@H](C)O2)C1)C(=O)c1c(OC)cccc1C3=O] | HA13 |
| 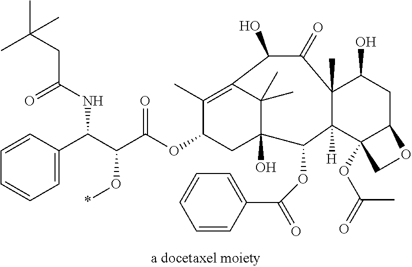 [SMILES: *O[C@@H](C(=O)O[C@H]1C[C@@]2(O)[C@@H](OC(=O)c3ccccc3)[C@@H]3[C@]4(OC(C)=O)CO[C@@H]4C[C@H](O)[C@@]3(C)C(=O)[C@H](O)C(=C1C)C2(C)C)[C@@H](NC(=O)CC(C)(C)C)c1ccccc1] | HA14 |
| 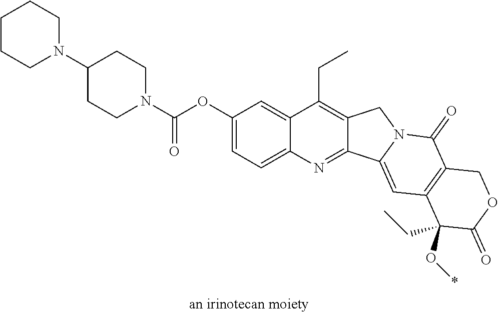 [SMILES: *O[C@]1(CC)C(=O)OCc2c1cc1n(c2=O)Cc2c-1nc1ccc(OC(=O)N3CCC(N4CCCCC4)CC3)cc1c2CC] | HA15 |
| 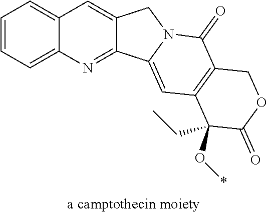 [SMILES: *O[C@]1(CC)C(=O)OCc2c1cc1n(c2=O)Cc2cc3ccccc3nc2-1] | HA16 |
| 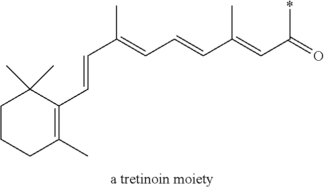 [SMILES: *C(=O)/C=C(C)/C=C/C=C(C)/C=C/C1=C(C)CCCC1(C)C] | HA17 |
| 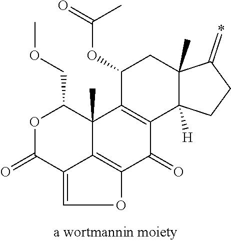 [SMILES: *=C1CC[C@H]2C3=C([C@H](OC(C)=O)C[C@]12C)[C@]1(C)c2c(coc2C3=O)C(=O)O[C@@H]1COC] | HA18 |
| 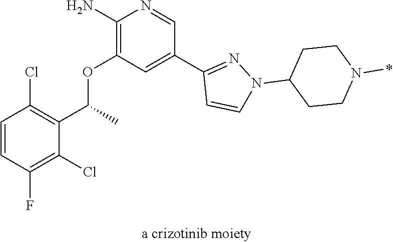 [SMILES: *N1CCC(n2ccc(-c3cnc(N)c(O[C@H](C)c4c(Cl)ccc(F)c4Cl)c3)n2)CC1] | HA19 |
| 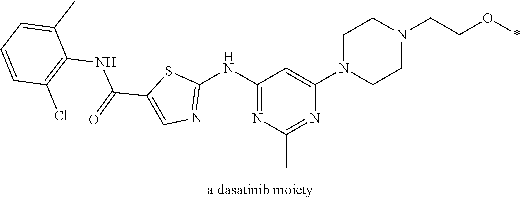 [SMILES: *OCCN1CCN(c2cc(Nc3ncc(C(=O)Nc4c(C)cccc4Cl)s3)nc(C)n2)CC1] | HA20 |
| 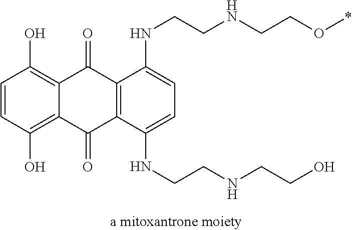 [SMILES: *OCCNCCNc1ccc(NCCNCCO)c2c1C(=O)c1c(O)ccc(O)c1C2=O] | HA21 |
| 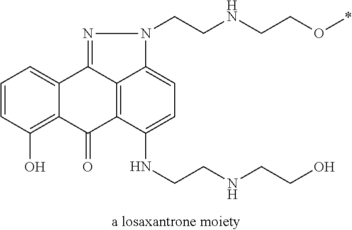 [SMILES: *OCCNCCn1nc2c3c(c(NCCNCCO)ccc31)C(=O)c1c(O)cccc1-2] | HA22 |
| 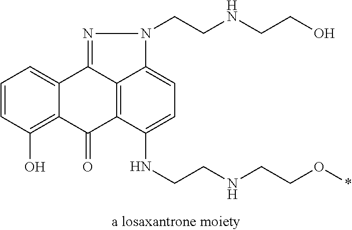 [SMILES: *OCCNCCNc1ccc2c3c(nn2CCNCCO)-c2cccc(O)c2C(=O)c13] | HA23 |
| 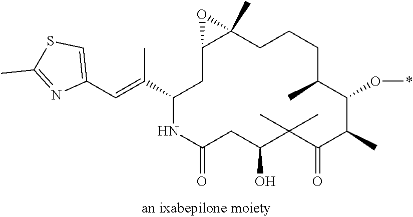 [SMILES: *O[C@H]1[C@@H](C)CCC[C@@]2(C)O[C@H]2C[C@@H](/C(C)=C/c2csc(C)n2)NC(=O)C[C@H](O)C(C)(C)C(=O)[C@@H]1C] | HA24 |

TABLE 2

| | -X²-X¹-A¹ Moieties |
|---|---|
| HB1 | —C(═O)—(CH₂)₁₄—C(═O)—OH |
| HB2 | —C(═O)—(CH₂)₁₆—C(═O)—OH |
| HB3 | —C(═O)—(CH₂)₁₈—C(═O)—OH |
| HB4 | —C(═O)—(CH₂)₇—CH═CH—(CH₂)₇—C(═O)—OH |
| HB5 | —C(═O)—(CH₂)₁₄—C(═O)—O—CH₃ |
| HB6 | —C(═O)—(CH₂)₁₆—C(═O)—O—CH₃ |
| HB7 | —C(═O)—(CH₂)₁₈—C(═O)—O—CH₃ |
| HB8 | —C(═O)—(CH₂)₇—CH═CH—(CH₂)₇—C(═O)—O—CH₃ |
| HB9 | —C(═O)—(CH₂)₁₄—CH₃ |
| HB10 | —C(═O)—(CH₂)₁₆—CH₃ |
| HB11 | —C(═O)—(CH₂)₁₈—CH₃ |
| HB12 | —C(═O)—(CH₂)₇—CH═CH—(CH₂)₇—CH₃ |
| HB13 | —C(═O)—(CH₂)₂—C(═O)—O—(CH₂)₁₅—C(═O)—OH |
| HB14 | —C(═O)—(CH₂)₂—C(═O)—O—(CH₂)₁₇—C(═O)—OH |
| HB15 | —C(═O)—(CH₂)₂—C(═O)—O—(CH₂)₁₉—C(═O)—OH |
| HB16 | —C(═O)—(CH₂)₂—C(═O)—O—(CH₂)₈—CH═CH—(CH₂)₇—C(═O)—OH |
| HB17 | —C(═O)—CH₂—NH—C(═O)—(CH₂)₁₄—C(═O)—OH |
| HB18 | —C(═O)—CH₂—NH—C(═O)—(CH₂)₁₆—C(═O)—OH |
| HB19 | —C(═O)—CH₂—NH—C(═O)—(CH₂)₁₈—C(═O)—OH |
| HB20 | —C(═O)—CH₂—NH—C(═O)—(CH₂)₇—CH═CH—(CH₂)₇—C(═O)—OH |
| HB21 | —C(═O)—(CH₂)₂—C(═O)—O—[(CH₂)₂—O—]₆C(═O)—(CH₂)₁₄—C(═O)—OH |
| HB22 | —C(═O)—(CH₂)₂—C(═O)—O—[(CH₂)₂—O—]₆C(═O)—(CH₂)₁₆—C(═O)—OH |
| HB23 | —C(═O)—(CH₂)₂—C(═O)—O—[(CH₂)₂—O—]₆C(═O)—(CH₂)₁₈—C(═O)—OH |
| HB24 | —C(═O)—(CH₂)₂—C(═O)—O—[(CH₂)₂—O—]₆C(═O)—(CH₂)₇—CH═CH—(CH₂)₇—C(═O)—OH |
| HB25 | —C(═O)—O—(CH₂)₁₅—C(═O)—OH |
| HB26 | —C(═O)—O—(CH₂)₁₇—C(═O)—OH |
| HB27 | —C(═O)—O—(CH₂)₁₉—C(═O)—OH |
| HB28 | —C(═O)—O—(CH₂)₈—CH═CH—(CH₂)₇—C(═O)—OH |
| HB29 | —C(═O)—NH—(CH₂)₁₅—C(═O)—OH |
| HB30 | —C(═O)—NH—(CH₂)₁₇—C(═O)—OH |
| HB31 | —C(═O)—NH—(CH₂)₁₉—C(═O)—OH |
| HB32 | —C(═O)—NH—(CH₂)₈—CH═CH—(CH₂)₇—C(═O)—OH |
| HB33 | —O—(CH₂)₁₅—C(═O)—OH |
| HB34 | —O—(CH₂)₁₇—C(═O)—OH |
| HB35 | —O—(CH₂)₁₉—C(═O)—OH |
| HB36 | —O—(CH₂)₈—CH═CH—(CH₂)₇—C(═O)—OH |
| HB37 | —NH—(CH₂)₂—O—C(═O)—(CH₂)₁₄—C(═O)—OH |
| HB38 | —NH—(CH₂)₂—O—C(═O)—(CH₂)₁₆—C(═O)—OH |
| HB39 | —NH—(CH₂)₂—O—C(═O)—(CH₂)₁₈—C(═O)—OH |
| HB40 | —NH—(CH₂)₂—O—C(═O)—(CH₂)₇—CH═CH—(CH₂)₇—C(═O)—OH |
| HB41 | —O—(CH₂)₂—O—C(═O)—(CH₂)₁₄—C(═O)—OH |
| HB42 | —O—(CH₂)₂—O—C(═O)—(CH₂)₁₆—C(═O)—OH |
| HB43 | —O—(CH₂)₂—O—C(═O)—(CH₂)₁₈—C(═O)—OH |
| HB44 | —O—(CH₂)₂—O—C(═O)—(CH₂)₇—CH═CH—(CH₂)₇—C(═O)—OH |
| HB45 | —NH—CH₂—C(═O)—O—[(CH₂)₂—O—]₆C(═O)—(CH₂)₁₄—C(═O)—OH |
| HB46 | —NH—CH₂—C(═O)—O—[(CH₂)₂—O—]₆C(═O)—(CH₂)₁₆—C(═O)—OH |
| HB47 | —NH—CH₂—C(═O)—O—[(CH₂)₂—O—]₆C(═O)—(CH₂)₁₈—C(═O)—OH |
| HB48 | —NH—CH₂—C(═O)—O—[(CH₂)₂—O—]₆C(═O)—(CH₂)₇—CH═CH—(CH₂)₇—C(═O)—OH |
| HB49 | —NH—(CH₂)₂—O—C(═O)—(CH₂)₁₄—C(═O)—O—CH₃ |
| HB50 | —NH—(CH₂)₂—O—C(═O)—(CH₂)₁₆—C(═O)—O—CH₃ |
| HB51 | —NH—(CH₂)₂—O—C(═O)—(CH₂)₁₈—C(═O)—O—CH₃ |
| HB52 | —NH—(CH₂)₂—O—C(═O)—(CH₂)₇—CH═CH—(CH₂)₇—C(═O)—O—CH₃ |
| HB53 | —CH₂—O—C(═O)—(CH₂)₁₄—C(═O)—OH |
| HB54 | —CH₂—O—C(═O)—(CH₂)₁₆—C(═O)—OH |
| HB55 | —CH₂—O—C(═O)—(CH₂)₁₈—C(═O)—OH |
| HB56 | —CH₂—O—C(═O)—(CH₂)₇—CH═CH—(CH₂)₇—C(═O)—OH |
| HB57 | —CH₂—O—C(═O)—(CH₂)₁₄—C(═O)—O—CH₃ |
| HB58 | —CH₂—O—C(═O)—(CH₂)₁₆—C(═O)—O—CH₃ |
| HB59 | —CH₂—O—C(═O)—(CH₂)₁₈—C(═O)—O—CH₃ |
| HB60 | —CH₂—O—C(═O)—(CH₂)₇—CH═CH—(CH₂)₇—C(═O)—O—CH₃ |
| HB61 | —CH₂—O—C(═O)—(CH₂)₁₄—CH₃ |
| HB62 | —CH₂—O—C(═O)—(CH₂)₁₆—CH₃ |
| HB63 | —CH₂—O—C(═O)—(CH₂)₁₈—CH₃ |
| HB64 | —CH₂—O—C(═O)—(CH₂)₇—CH═CH—(CH₂)₇—CH₃ |
| HB65 | —CH₂—O—C(═O)—CH₂—NH—C(═O)—(CH₂)₁₄—C(═O)—OH |
| HB66 | —CH₂—O—C(═O)—CH₂—NH—C(═O)—(CH₂)₁₆—C(═O)—OH |
| HB67 | —CH₂—O—C(═O)—CH₂—NH—C(═O)—(CH₂)₁₈—C(═O)—OH |
| HB68 | —CH₂—O—C(═O)—CH₂—NH—C(═O)—(CH₂)₇—CH═CH—(CH₂)₇—C(═O)—OH |
| HB69 | —CH₂—O—C(═O)—(CH₂)₂—C(═O)—O—[(CH₂)₂—O—]₆C(═O)—(CH₂)₁₄—C(═O)—OH |
| HB70 | —CH₂—O—C(═O)—(CH₂)₂—C(═O)—O—[(CH₂)₂—O—]₆C(═O)—(CH₂)₁₆—C(═O)—OH |
| HB71 | —CH₂—O—C(═O)—(CH₂)₂—C(═O)—O—[(CH₂)₂—O—]₆C(═O)—(CH₂)₁₈—C(═O)—OH |
| HB72 | —CH₂—O—C(═O)—(CH₂)₂—C(═O)—O—[(CH₂)₂—O—]₆C(═O)—(CH₂)₇—CH═CH—(CH₂)₇—C(═O)—OH |
| HB73 | —CH₂—O—C(═O)—O—(CH₂)₁₅—C(═O)—OH |
| HB74 | —CH₂—O—C(═O)—O—(CH₂)₁₇—C(═O)—OH |
| HB75 | —CH₂—O—C(═O)—O—(CH₂)₁₉—C(═O)—OH |
| HB76 | —CH₂—O—C(═O)—O—(CH₂)₈—CH═CH—(CH₂)₇—C(═O)—OH |
| HB77 | —CH₂—O—C(═O)—NH—(CH₂)₁₅—C(═O)—OH |
| HB78 | —CH₂—O—C(═O)—NH—(CH₂)₁₇—C(═O)—OH |
| HB79 | —CH₂—O—C(═O)—NH—(CH₂)₁₉—C(═O)—OH |
| HB80 | —CH₂—O—C(═O)—NH—(CH₂)₈—CH═CH—(CH₂)₇—C(═O)—OH |
| HB81 | ═N—O—(CH₂)₁₅—C(═O)—OH |
| HB82 | ═N—O—(CH₂)₁₇—C(═O)—OH |
| HB83 | ═N—O—(CH₂)₁₉—C(═O)—OH |
| HB84 | ═N—O—(CH₂)₈—CH═CH—(CH₂)₇—C(═O)—OH |
| HB85 | ═N—NH—(CH₂)₁₅—C(═O)—OH |
| HB86 | ═N—NH—(CH₂)₁₇—C(═O)—OH |
| HB87 | ═N—NH—(CH₂)₁₉—C(═O)—OH |
| HB88 | ═N—NH—(CH₂)₈—CH═CH—(CH₂)₇—C(═O)—OH |
| HB89 | ═N—O—[(CH₂)₂—O—]₆(CH₂)₁₅—C(═O)—OH |
| HB90 | ═N—O—[(CH₂)₂—O—]₆(CH₂)₁₇—C(═O)—OH |
| HB91 | ═N—O—[(CH₂)₂—O—]₆(CH₂)₁₉—C(═O)—OH |
| HB92 | ═N—O—[(CH₂)₂—O—]₆(CH₂)₈—CH═CH—(CH₂)₇—C(═O)—OH |
| HB93 | —C(═O)—CH₂—O—C(═O)—(CH₂)₁₄—C(═O)—OH |
| HB94 | —C(═O)—CH₂—O—C(═O)—(CH₂)₁₆—C(═O)—OH |
| HB95 | —C(═O)—CH₂—O—C(═O)—(CH₂)₁₈—C(═O)—OH |
| HB96 | —C(═O)—CH₂—O—C(═O)—(CH₂)₇—CH═CH—(CH₂)₇—C(═O)—OH |
| HB97 | —C(═O)—CH(CH₃)—O—C(═O)—(CH₂)₁₄—C(═O)—OH |
| HB98 | —C(═O)—CH(CH₃)—O—C(═O)—(CH₂)₁₆—C(═O)—OH |
| HB99 | —C(═O)—CH(CH₃)—O—C(═O)—(CH₂)₁₈—C(═O)—OH |
| HB100 | —C(═O)—CH(CH₃)—O—C(═O)—(CH₂)₇—CH═CH—(CH₂)₇—C(═O)—OH |
| HB101 | —C(═O)—(CH₂)₅—O—C(═O)—(CH₂)₁₄—C(═O)—OH |
| HB102 | —C(═O)—(CH₂)₅—O—C(═O)—(CH₂)₁₆—C(═O)—OH |
| HB103 | —C(═O)—(CH₂)₅—O—C(═O)—(CH₂)₁₈—C(═O)—OH |
| HB104 | —C(═O)—(CH₂)₅—O—C(═O)—(CH₂)₇—CH═CH—(CH₂)₇—C(═O)—OH |

TABLE 3

| Compound No. | A²- Moiety | -X²-X¹-A¹ Moiety |
|---|---|---|
| 1-44 | HA1 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 45-88 | HA2 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 89-132 | HA9 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 133-176 | HA12 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 177-220 | HA14 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 221-264 | HA15 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 265-308 | HA16 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 309-352 | HA19 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 353-396 | HA20 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 397-440 | HA21 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 441-484 | HA22 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 485-528 | HA23 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 529-572 | HA24 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, respectively |
| 573-592 | HA5 | HB33, HB34, HB35, HB36, HB37, HB38, HB39, |
| | | HB40, HB41, HB42, HB43, HB44, HB45, HB46, |
| | | HB47, HB48, HB49, HB50, HB51, HB52, |
| | | respectively |
| 593-612 | HA6 | HB33, HB34, HB35, HB36, HB37, HB38, HB39, |
| | | HB40, HB41, HB42, HB43, HB44, HB45, HB46, |
| | | HB47, HB48, HB49, HB50, HB51, HB52, |
| | | respectively |
| 613-632 | HA7 | HB33, HB34, HB35, HB36, HB37, HB38, HB39, |
| | | HB40, HB41, HB42, HB43, HB44, HB45, HB46, |
| | | HB47, HB48, HB49, HB50, HB51, HB52, |
| | | respectively |
| 633-652 | HA8 | HB33, HB34, HB35, HB36, HB37, HB38, HB39, |
| | | HB40, HB41, HB42, HB43, HB44, HB45, HB46, |
| | | HB47, HB48, HB49, HB50, HB51, HB52, |
| | | respectively |
| 653-672 | HA11 | HB33, HB34, HB35, HB36, HB37, HB38, HB39, |
| | | HB40, HB41, HB42, HB43, HB44, HB45, HB46, |
| | | HB47, HB48, HB49, HB50, HB51, HB52, |
| | | respectively |
| 673-692 | HA17 | HB33, HB34, HB35, HB36, HB37, HB38, HB39, |
| | | HB40, HB41, HB42, HB43, HB44, HB45, HB46, |
| | | HB47, HB48, HB49, HB50, HB51, HB52, |
| | | respectively |
| 693-720 | HA3 | HB53, HB54, HB55, HB56, HB57, HB58, HB59, |
| | | HB60, HB61, HB62, HB63, HB64, HB65, HB66, |
| | | HB67, HB68, HB69, HB70, HB71, HB72, HB73, |
| | | HB74, HB75, HB76, HB77, HB78, HB79, HB80, |
| | | respectively |
| 721-748 | HA4 | HB53, HB54, HB55, HB56, HB57, HB58, HB59, |
| | | HB60, HB61, HB62, HB63, HB64, HB65, HB66, |
| | | HB67, HB68, HB69, HB70, HB71, HB72, HB73, |
| | | HB74, HB75, HB76, HB77, HB78, HB79, HB80, |
| | | respectively |
| 749-760 | HA10 | HB81, HB82, HB83, HB84, HB85, HB86, HB87, |
| | | HB88, HB89, HB90, HB91, HB92, respectively |
| 761-772 | HA13 | HB81, HB82, HB83, HB84, HB85, HB86, HB87, |
| | | HB88, HB89, HB90, HB91, HB92, respectively |
| 773-784 | HA18 | HB81, HB82, HB83, HB84, HB85, HB86, HB87, |
| | | HB88, HB89, HB90, HB91, HB92, respectively |

The foregoing compounds may be formulated into pharmaceutical compositions in any suitable manner. In general, as compounds for the treatment of cancer, such pharmaceutical formulations are aqueous formulations suitable for parenteral administration, such as intravenous or intra-arterial administration.

In at least one aspect, the disclosure uses of the cytotoxic prodrug in pharmaceutical compositions that include one or more compounds of formula (I) (according to any of the foregoing embodiments) and a protein. In some embodiments, the protein is an albumin or an albumin mimetic. In some such embodiments, the protein is human serum albumin (HSA) or a mimetic thereof, i.e., a protein whose sequence is at least 50% equivalent to that of HSA, or at least 60% equivalent to that of HSA, or at least 70% equivalent to that of HSA, or at least 80% equivalent to that of HSA, or at least 90% equivalent to that of HSA, or at least 95% equivalent to that of HSA, at least 97% equivalent to that of HSA, at least 99% equivalent to that of HSA. In some embodiments, the protein is human serum albumin.

In certain embodiments of any of the foregoing embodiments, the pharmaceutical composition also includes a carrier, such as a liquid carrier. In some embodiments, the carrier includes water. For example, in some such embodiments, water makes up at least 50% by volume, or at least 60% by volume, or at least 70% by volume, or at least 80% by volume, or at least 90% by volume, based on the total volume of liquid materials in the pharmaceutical composition. The carrier can also include other liquid ingredients, such as liquid ingredients commonly included in aqueous pharmaceutical formulations for parenteral administration.

In certain embodiments having an aqueous carrier, the compounds of formula (I) bind non-covalently to the protein in the pharmaceutical formulation. In some embodiments, the compound of formula (I) and the protein (e.g., human serum albumin) are non-covalently associated with each other with a binding constant (K_{b}) of at least 10² M⁻¹, or at least 10³ M⁻¹, or at least 10⁴ M⁻¹, or at least 10⁵ M⁻¹ at 25° C. in the aqueous composition.

In some embodiments having an aqueous carrier, the compound of formula (I) and the protein are solvated by the carrier. In some such embodiments, at least 90% by weight, or at least 95% by weight, or at least 97% by weight, or at least 98% by weight, or at least 99% by weight of the compounds of formula (I) in the composition are bound non-covalently to the protein with a binding constant (K_{b}) of at least 10² M⁻¹, or at least 10³ M⁻¹, or at least 10⁴ M⁻¹, or at least 10⁵ M⁻¹ at 25° C. in the aqueous composition. In some further such embodiments, the composition is substantially free of agglomerates or nanoparticles. For example, in some embodiments of any of the aforementioned embodiments, no more than 5% by weight, or no more than 4% by weight, or no more than 3% by weight, or no more than 2% by weight, or no more than 1% by weight of the protein-compound (i.e., non-covalently bound conjugates between the protein and one or more compounds of formula (I)) in the aqueous composition have a radius greater than 7 nm, or a radius greater than 5 nm, or a radius greater than 4 nm, as measured by dynamic light scattering.

The compound of formula (I) can have any suitable molar ratio to the protein in the formulation. For example, in some embodiments of any of the foregoing embodiments, the molar ratio of the compound of formula (I) to the protein ranges from 1:10 to 20:1, or from 1:5 to 15:1, or from 1:2 to 10:1. In some embodiments of any of the foregoing embodiments, the molar ratio of the compound of formula (I) to the protein is about 1:1, or is about 2:1, or is about 3:1, or is about 4:1, or is about 5:1, or is about 6:1, or is about 7:1, wherein the term “about,” in this instance means ±0.5:1, such that “about 5:1” refers to a range from 4.5:1 to 5.5:1.

In general, the pharmaceutical compositions used herein include: a compound, which comprises a cytotoxic drug moiety and a protein binding moiety; a protein, wherein the protein is an albumin or an albumin mimetic; and a carrier, which comprises water.

In some embodiments, the protein is human serum albumin (HSA) or a mimetic thereof, i.e., a protein whose sequence is at least 50% equivalent to that of HSA, or at least 60% equivalent to that of HSA, or at least 70% equivalent to that of HSA, or at least 80% equivalent to that of HSA, or at least 90% equivalent to that of HSA, or at least 95% equivalent to that of HSA, at least 97% equivalent to that of HSA, at least 99% equivalent to that of HSA. In some embodiments, the protein is human serum albumin.

As noted above, in some embodiments, the carrier includes water. For example, in some such embodiments, water makes up at least 50% by volume, or at least 60% by volume, or at least 70% by volume, or at least 80% by volume, or at least 90% by volume, based on the total volume of liquid materials in the pharmaceutical composition. The carrier can also include other liquid ingredients, such as liquid ingredients commonly included in aqueous pharmaceutical formulations for parenteral administration.

In certain embodiments, the compounds bind non-covalently to the protein in the pharmaceutical formulation. In some embodiments, the compound and the protein (e.g., human serum albumin) are non-covalently associated with each other with a binding constant (K_{b}) of at least 10² M⁻¹, or at least 10³ M⁻¹, or at least 10⁴ M⁻¹, or at least 10⁵ M⁻¹ at 25° C. in the aqueous composition.

In some embodiments having an aqueous carrier, the compound and the protein are solvated by the carrier. In some such embodiments, at least 90% by weight, or at least 95% by weight, or at least 97% by weight, or at least 98% by weight, or at least 99% by weight of the compounds of formula (I) in the composition are bound non-covalently to the protein with a binding constant (K_{b}) of at least 10² M⁻¹, or at least 10³ M⁻¹, or at least 10⁴ M⁻¹, or at least 10⁵ M⁻¹ at 25° C. in the aqueous composition. In some further such embodiments, the composition is substantially free of agglomerates or nanoparticles. For example, in some embodiments of any of the aforementioned embodiments, no more than 5% by weight, or no more than 4% by weight, or no more than 3% by weight, or no more than 2% by weight, or no more than 1% by weight of the protein-compound (i.e., non-covalently bound conjugates between the protein and one or more compounds of formula (I)) in the aqueous composition have a radius greater than 7 nm, or a radius greater than 5 nm, or a radius greater than 4 nm, as measured by dynamic light scattering.

The compound of formula (I) can have any suitable molar ratio to the protein in the formulation. For example, in some embodiments of any of the foregoing embodiments, the molar ratio of the compound of formula (I) to the protein ranges from 1:10 to 20:1, or from 1:5 to 15:1, or from 1:2 to 10:1. In some embodiments of any of the foregoing embodiments, the molar ratio of the compound of formula (I) to the protein is about 1:1, or is about 2:1, or is about 3:1, or is about 4:1, or is about 5:1, or is about 6:1, or is about 7:1, wherein the term “about,” in this instance means ±0.5:1, such that “about 5:1” refers to a range from 4.5:1 to 5.5:1.

The pharmaceutical compositions of any of the foregoing aspects and embodiments can also include certain additional ingredients, such as those commonly employed in pharmaceutical compositions for parenteral administration, such as excipients commonly used in fluids suitable for intravenous administration. Non-limiting examples of additional components include water, various salts (such as sodium chloride), various sugars (such as glucose, dextrose, and the like), pH buffers, thickeners (such as cellulosic materials), amino acids, proteins, vitamins and other nutrients, anti-nausea agents, preservatives, surfactants, emulsifiers, and the like.

Such pharmaceutical compositions may be administered according to any suitable dosage regimen and in any suitable amount. When administered by intravenous infusion, for example, the compound of formula (I) is administered as a dose of 50-1000 mg/m² over the course of 1 to 48 hours. In some further embodiments, the compound of formula (I) is administered as a dose of 50-500 mg/m² over the course of 1 to 24 hours. Such infusions can be part of a multi-dose regimen. For example, following an initial administration at the quantities set forth above, the subject is administered a similar dose on one, two, three, four, five, six, or more additional occasions. Such additional administrations may be separated from each other for any suitable time period, such as one week, two weeks, three weeks, four weeks, five weeks, or six weeks.

In the methods and uses disclosed herein, the foregoing compounds and compositions are administered to a subject for the treatment of cancer and related disorders. In some embodiments, these compounds and compositions are be used for administration to a subject who has or has had a cancerous tumor. In some embodiments, the cancer is a cancer that comprises cells that overexpress fatty acid uptake proteins. In some further embodiments, the cancer is a cancer that comprises cells that overexpress CD36. As used herein, the term “overexpress” refers to an abnormally high expression of a particular protein or class of proteins relative to healthy cells of a similar type. For example, in the case of certain breast cancers, the cells of the tumor may be said to overexpress certain proteins because the cancerous cells express certain proteins to a greater degree than the cells of normal, healthy breast tissue. The overexpression can be of any suitable degree. For example, in some embodiments, the cancer comprises cells that overexpress fatty acid uptake proteins (or, in some embodiments, CD36) at a concentration of at least 10% more, or at least 25% more, or at least 50% more, or at least 100% more than the cells of normal, healthy tissue of the infected tissue type.

A variety of cancers may, in the course of their evolution, overexpress certain fatty acid uptake proteins, such as CD36. In particular, overexpression of fatty acid uptake proteins may occur in metastatic cancers. Thus, in some embodiments, the cancer being treated is a metastatic cancer, such as a metastatic cancerous tumor.

Such tumors can affect various systems of the body. Thus, the cancers being treated include, but are not limited to: cancers of epithelial origin, such as breast cancer, prostate cancer, ovarian cancer, and colon cancer; hepatic carcinomas and gliomas; gastric cancer; glioblastomas; oral carcinomas, such as oral squamous cell carcinoma; acute myeloid leukemia; lung squamous cell carcinomas; and bladder cancer. Various cancers in which CD36 expression plays a role are set forth in Enciu et al., BIOMED. RES. INT., vol. 2018:7801202 (published online Jul. 4, 2018), the contents of which are hereby incorporated by reference.

In some embodiments, the cancer is a metastatic cancer, such as a metastatic tumor. In some further embodiments, the cancer is metastatic breast cancer. In some other embodiments, the cancer is metastatic prostate cancer. In some other embodiments, the cancer is metastatic ovarian cancer. In some other embodiments, the cancer is metastatic colon cancer. In some other embodiments, the cancer is metastatic hepatic cancer, such as a metastatic hepatic carcinoma or glioma. In some embodiments, the compounds of formula (I) are administered in combination with one or more other compounds. In some embodiments, the one or more other compounds includes another cytotoxin, such as a small-molecule cytotoxin. In some other embodiments, the one or more other compounds comprises one or more oligonucleotide compounds.

The administration of compounds of formula (I) can also be included within a dosage regimen that includes administering one or more additional chemotherapeutic agents to the subject. Non-limiting examples of such additional chemotherapeutic agents include, but are not limited to taxanes, topoisomerase I inhibitors, topoisomerase II inhibitors, alkylating agents, anthracycline compounds, platinum-based compounds, anti-folate compounds, purine analogs, vinca alkaloids, kinase inhibitors, ubiquitin ligase modulators, androgen receptor agonists, proteasome inhibitors, Hedgehog signaling pathway modulators, epothilone compounds, cytotoxic oligonucleotides, cyctotoxic proteins, and the like.

In certain embodiments, the compounds of formula (I) are used to “prime” the cancer and improve the potential efficacy of immunomodulating agents. Thus, in some embodiments, the compounds of formula (I) are administered in combination with one or more immunomodulating agents. Any suitable immunomodulating agents can be used, including, but not limited to: monoclonal antibodies (such as alemtuzumab, atezolizumab, ipilimumab, nivolumab, and pembrolizumab), anti-CD47 antibodies, anti-SIRP-alpha antibodies, anti-GD2 antibodies, anti-PD-1 antibodies, anti-PD-Ll antibodies, immune checkpoint inhibitors (such as CTLA-4 inhibitors), and toll-like receptor (TLR) agonists Thus, the administration of a compound of formula (I) can, in some embodiments, be part of a dosage regimen that includes administering one or more immunomodulating agents. For example, in certain embodiments, the subject is treated initially with a dosage (or dosage regimen) of a compound of formula (I) followed by a subsequent administration with one or more immunomodulating compounds.

### EXAMPLES

The following examples show certain illustrative embodiments of the compounds, compositions, and methods disclosed herein. These examples are not to be taken as limiting in any way. Nor should the examples be taken as expressing any preferred embodiments, or as indicating any direction for further research.

The examples may use abbreviations for certain common chemicals. The following abbreviations refer to the compounds indicated.

DMF=Dimethylformamide

DCM=Dichloromethane

NMR=Nuclear magnetic resonance

HPLC=High-performance liquid chromatography

RP-HLPC=Reverse-phase high-performance liquid chromatography

LRMS=Liquid chromatography/low-resolution mass spectrometry

HRMS=Liquid chromatography/high-resolution mass spectrometry

Tips=Triisopropylsilyl

DMAP=4-(Dimethylamino)pyridine

EDC=1-Ethyl-3-(3-dimethylaminopropyl)carbodiimide

THF=Tetrahydrofuran

Dipea=N,N-diisopropylethylamine

HATU=1-[Bis(dimethylamino)methylene]-1H-1,2,3-triazolo-[4,5 -b]pyridinium 3-oxide hexafluorophosphate

DCC=N,N′-dicyclohexylcarbodiimide

HSA=Human serum albumin

### Example 1—Mono-Tips Protected C18 Diacid

To a solution of the diacid (10 g) in dry DMF (150 mL) at 60° C. was added triisopropylsilyl chloride (6.68 mL). To the stirred solution was added dropwise freshly distilled triethylamine (4.35 mL). The reaction was stirred overnight under nitrogen atmosphere then the solution was cooled to room temperature, filtered and concentrated to dryness. Dichloromethane (150 mL) was added to the solid and the round bottom sonicated. The solid was filtered off and the solvent was removed under vacuum. The residue was purified by column chromatography (2.5% THF in DCM) to give a clear oil. 6.1 g, 41% yield. LRMS—471.32 [M+H]⁺, HRMS—Theoretical=471.3864, Observed=471.3861. ¹H NMR (CDCl₃): δ(ppm) 1.06-1.09 (m, 21H), 1.2-1.4 (m, 24H), 1.55-1.70 (m, 4H), 2.30-2.40 (m, 4H).

### Example 2—Paclitaxel-C18 Diacid Conjugate (ODDA-PTX)

To a stirring solution of paclitaxel (500 mg) in dry DCM (50 mL) at 0° C. was added DMAP (231 mg). After 5 minutes, EDC (137 mg) was added. After an additional 5 minutes, was added Mono-Tips Protected C18 Diacid (791 mg) and the resulting solution allowed to stir, and warm to room temperature overnight. The crude reaction mixture was then transferred to a separatory funnel and washed with water (3×50 mL), saturated NaCl (3×50 mL) and 0.1M HCl (3×50 mL). The organic phase was dried over MgSO₄, and rotovapped to afford a yellowish-white solid. To remove the TIPS protecting group, the solid was taken up in THF and Bu₄NF (1.2 g, 0.0046 mol) was added and stirred. After 18 h, excess AMBERLITE ion exchange resin and excess CaCO₃ were added to the reaction mixture. After 1 h, the solution was filtered and the filtrate concentrated to a free-flowing oil. The oil was dissolved in 50 mL DCM, and upon addition of water a white precipitate crashed out. The reaction mixture was filtered and transferred to a separatory funnel, where it was extracted with water (3×50 mL). The organic phase was dried over MgSO₄, filtered, and concentrated to afford a clear/white, glassy solid. 521 mg, 81% yield. MS—Theoretical=1147.59 [M-H]⁻, Observed=1148.38. ¹H NMR (CDCl₃): δ(ppm) 0.08-1.10 (m, 21H), 1.2-1.4 (m, 24H), 1.55-1.64 (m, 4H), 1.65-1.7 (m, 2H), 1.8-1.95 (m, 4H), 1.95-1.05 (m, 2H), 2.1-2.15 (m, 4H), 2.25-2.40 (m, 6H), 2.4-2.5 (m, 2H), 2.5-2.55 (m, 1H), 3.7 (s, 1H), 3.75-3.95 (m, 3H), 4.1 (m, 1H), 4.2 (m, 1H), 4.3 (m, 1H), 4.4 (m, 1H), 4.7 (m, 1H), 4.95 (m, 1H), 5.5 (m, 1H), 5.7 (m, 1H), 5.8 (m, 1H), 6.2-6.3 (m, 2H), 6.8 (m, 1H), 6.97 (m, 1H), 7.05 (m, 1H), 7.3-7.45 (m, 5H), 7.45-7.55 (m, 4H), 7.6 (m, 1H), 7.75 (m, 2H), 8.1-8.2 (m, 2H).

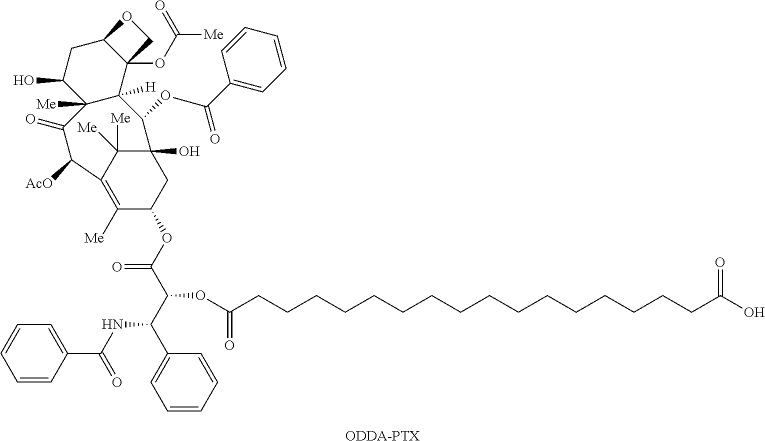

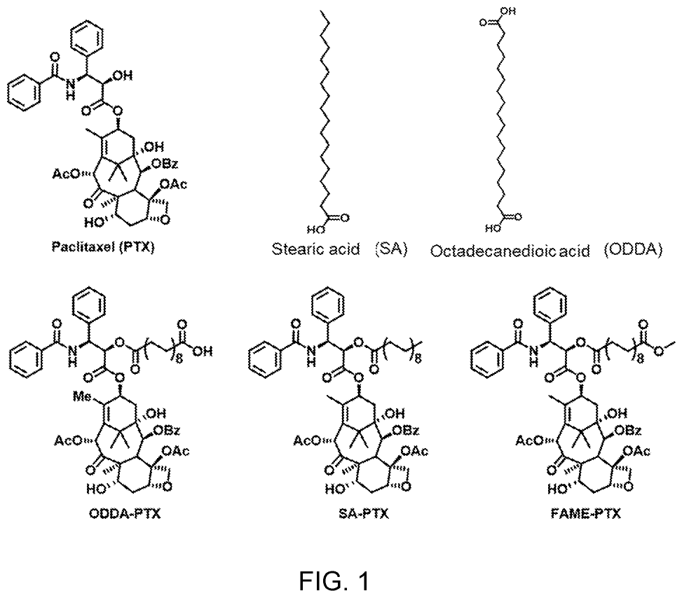

Additional PTX conjugates, as shown in FIG. 1, with, acids, diacids and esters thereof, for example, steric acid (SA-PTX) and with the methyl ester of octadecanoic acid (FAME-PTX) can be prepared by analogous methods.

### Example 3—Comparative Uptake

The compound of Example 2 (ODDA-PTX) was compared with paclitaxel (PTX) in terms of cellular uptake. The comparison was carried out in HT-1080 cell culture using sulfosuccinimidyl oleate (SSO), a chemical blocker of fatty acid translocase/CD36:

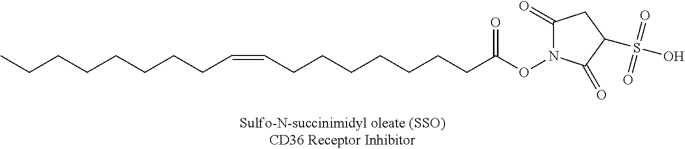 [SMILES: CCCCCCCC/C=C\CCCCCCCC(=O)ON1C(=O)CC(S(=O)(=O)O)C1=O]

Cytotoxicity of the compounds was evaluated using the CellTiter Blue (CTB) assay (Promega, cat G8081). Treatments of fatty acid-conjugates were prepared as 1000× serial stock dilutions in DMSO, then diluted into media for 1×, 0.1% DMSO treatment solutions. Likewise, treatments with SSO (Cayman Chemical, cat 11211) were prepared as 1000× serial stock dilutions in DMSO, then diluted into media with the final concentration of DMSO in solution varying but never exceeding 1.1% DMSO in media.

Expression of CD36 in HT-1080 was validated using flow cytometry and a fluorophore-conjugated CD36 antibody (CD36, eFluor 660, clone: eBioNL07 (NL07), eBioscience). Cells were harvested, washed, and resuspended at about 1×10⁶ cells/mL in ice-cold resuspension buffer (DPBS, supplemented with 10% fetal bovine serum+1% sodium azide). Then, 100 μL of cell suspension was aliquoted into Eppendorf tubes and 5 μL CD36 antibody (0.125 μg) was added to each vessel and incubated in the dark for 30 min. at 37° C. After incubation, cells were washed 3× with 500 μL of resuspension buffer. Cells were pelleted via centrifugation at 200× g for 5 minutes between each wash. Cells were resuspended in a final volume of 100 μL resuspension and analyzed via flow cytometry for presence of CD36. As a control, the same protocol was repeated, but without the antibody incubation.

HT-1080 cells were plated in 96-well plates one day before treatment, at a seeding density of 10,000 cells/well. After 24 hours, plating media was removed, then treatments of 100 μL were added to the wells. For those treated with SSO, cells were incubated with the media containing the respective concentration of blocker one hour prior to treatment. This initial treatment was removed before adding media containing therapeutic and additional blocker matching the concentration. This process was to ensure that there is additional blocker present as cells divide and produce more CD36 FAT instances. After three days, the media was removed and replaced with 100 μL complete DMEM without phenol red. Then, 20 μL of CTB reagent was added, and the cells were incubated for two hours at 37° C. Fluorescence was measured at 590 nm with excitation at 560 nm using a Perkin Elmer EnSpire plate reader. Average background fluorescence readings of the experimental wells (three wells per treatment concentration). Viability was calculated as the average background-subtracted signal in a well compared to that of a negative control (cells treated with vehicle, 0.1 to 1.1% DMSO in media). As a control, 20% DMSO in media was used. Statistical analysis was performed using an unpaired t-test, with p-values<0.5 considered statistically significant.

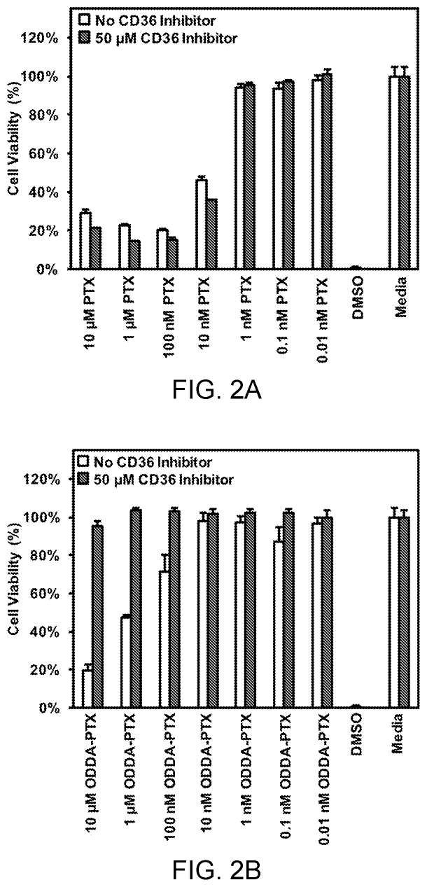

FIG. 2A and FIG. 2B show the change in cytotoxicity in HT-1080 cells as a function of CD36 inhibition. FIG. 2A shows the PTX cytotoxicity in the absence (black) or presence (white) of the CD36 inhibitory compound, SSO, at increasing PTX concentrations. FIG. 2B shows the ODDA-PTX cytotoxicity in the absence (black) or presence (white) of the CD36 inhibitory compound, SSO, at increasing PTX concentrations.

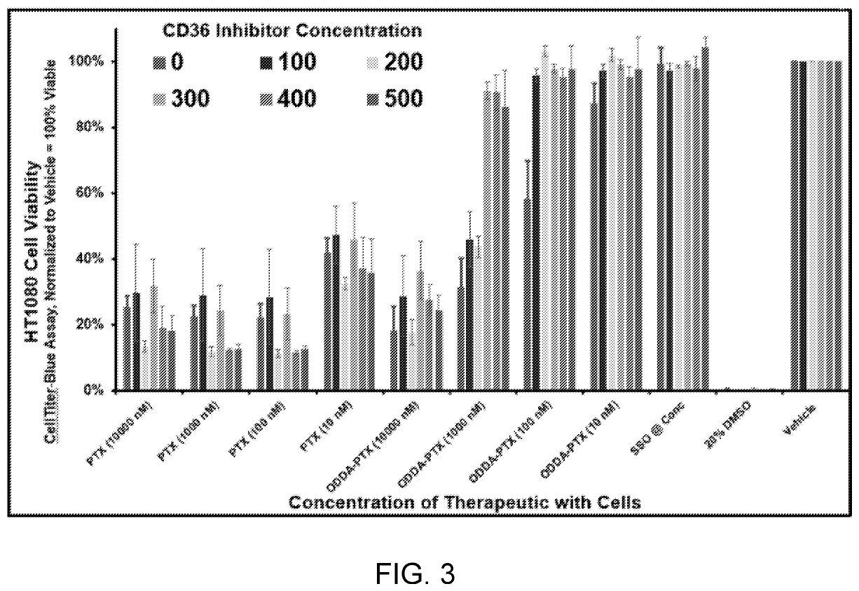

Further, such experiments were carried out using the HT-1080 cell line. FIG. 3 shows data for HT-1080 cells treated with both PTX and ODDA-PTX at different concentrations, and at different concentrations of SSO. The vertical axis shows percent viability of the cells. The data show that increasing the concentration of the CD36 inhibitor correlates with increasing percent viability for cells treated with ODDA-PTX but not for cells treated with PTX.

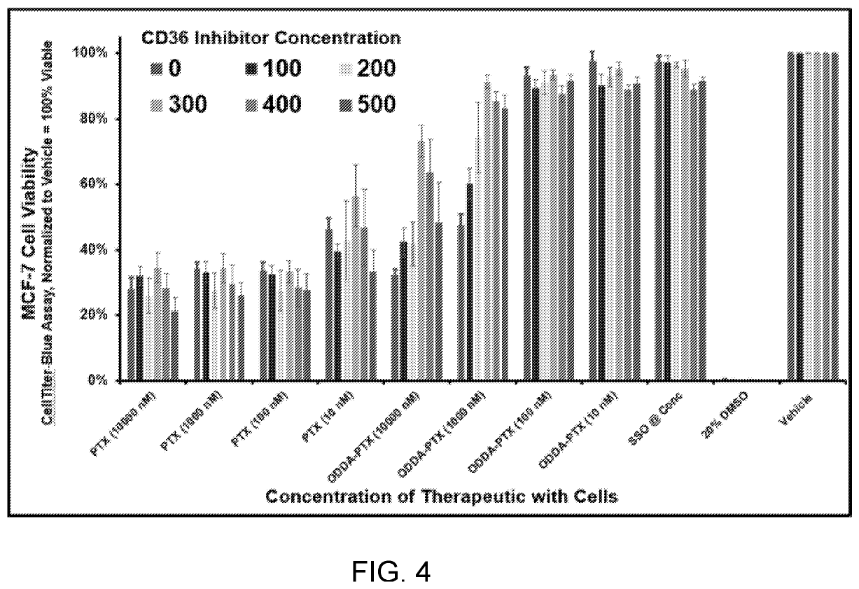

Further, such experiments were carried out using the MCF-7 cell line. FIG. 4 shows data for MCF-7 cells treated with both PTX and ODDA-PTX at different concentrations, and at different concentrations of SSO. The vertical axis shows percent viability of the cells. The data show that increasing the concentration of the CD36 inhibitor correlates with increasing percent viability for cells treated with ODDA-PTX but not for cells treated with PTX.

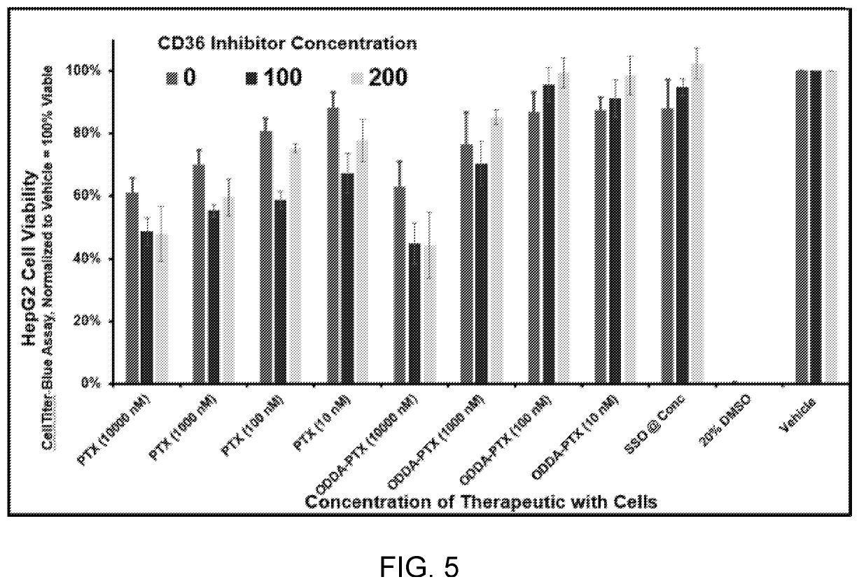

Further, such experiments were carried out using the HepG2 cell line. FIG. 5 shows data for HepG2 cells treated with both PTX and ODDA-PTX at different concentrations, and at different concentrations of SSO. The vertical axis shows percent viability of the cells. The data show that increasing the concentration of the CD36 inhibitor correlates with increasing percent viability for cells treated with ODDA-PTX but not for cells treated with PTX.

1. A method of treating cancer, the method comprising administering to a subject, in need of treatment for cancer, an effective amount of a compound of formula (I):
A¹-X¹-X²-A² (I)
or a pharmaceutically acceptable salt thereof;
wherein:
A¹ is a carboxylic acid group, a carboxylate anion, or a carboxylate ester;
A² is a cytotoxic drug moiety, which has a molecular weight of no more than 1600 Da;
X¹ is a hydrophobic group; and
X² is a direct bond, an organic group, —O—, —S—, —S(═O)—, —S(═O)₂—, —S—S—, —N═, ═N—, —N(H)—, —N═N—N(H)—, —N(H)—N═N—, —N(OH)—, or —N(═O)—; and
wherein the cancer comprises cells that overexpress one or more fatty acid uptake proteins.

2. The method of claim 1, wherein A¹ is a carboxylic acid group.

3. The method of claim 1, wherein the cytotoxic drug moiety is a taxane moiety.

4. The method of claim 3, wherein the taxane moiety is a paclitaxel moiety or a docetaxel moiety.

5. (canceled)

6. The method of claim 4, wherein the taxane moiety is a paclitaxel moiety and the paclitaxel moiety is a moiety of the formula:

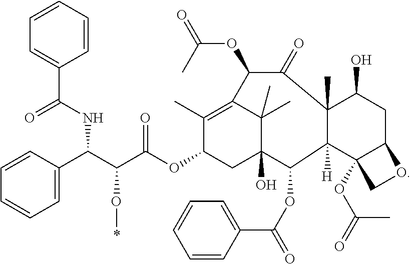 [SMILES: *O[C@@H](C(=O)O[C@H]1C[C@@]2(O)[C@@H](OC(=O)c3ccccc3)[C@@H]3[C@]4(OC(C)=O)CO[C@@H]4C[C@H](O)[C@@]3(C)C(=O)[C@H](OC(C)=O)C(=C1C)C2(C)C)[C@@H](NC(=O)c1ccccc1)c1ccccc1]

7. The method of claim 1, wherein X¹ is C₁₂₋₂₂ hydrocarbylene, which is optionally substituted.

8. The method of claim 1, which is a compound of the formula: 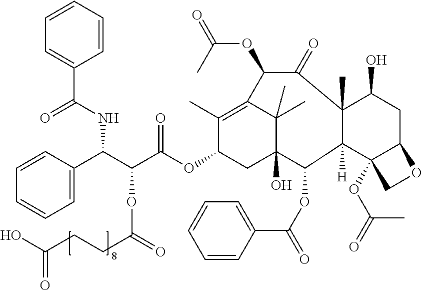
or a pharmaceutically acceptable salt thereof.

9. The method of claim 1, wherein the subject is a human.

10.-11. (canceled)

12. The method of claim 1, wherein the administration comprises parenteral administration.

13. The method of claim 12, wherein the parenteral administration comprises intramuscular administration, intravenous administration, or subcutaneous administration.

14. (canceled)

15. The method of claim 1, wherein the compound of formula (I) is formulated as a pharmaceutical composition that further comprises a carrier.

16. The method of claim 15, wherein the pharmaceutical composition further comprises a protein, wherein the protein is human serum albumin or a protein whose sequence is at least 50% equivalent to that of human serum albumin.

17. The method of claim 16, wherein the protein is human serum albumin.

18. The method of claim 15, wherein the carrier comprises water.

19. The method of claim 1, wherein the compound of formula (I) is administered in combination with one or more additional chemotherapeutic agents.

20. The method of claim 19, wherein the one or more additional chemotherapeutic agents comprise one of more compounds selected from the group consisting of: taxanes, topoisomerase I inhibitors, topoisomerase II inhibitors, alkylating agents, anthracycline compounds, platinum-based compounds, anti-folate compounds, purine analogs, vinca alkaloids, kinase inhibitors, ubiquitin ligase modulators, androgen receptor agonists, proteasome inhibitors, Hedgehog signaling pathway modulators, epothilone compounds, cytotoxic oligonucleotides, cytotoxic proteins.

21. The method of claim 1, wherein the compound of formula (I) is administered in combination with one or more immunomodulating agents.

22. The method of claim 21, wherein the one or more immunomodulating agents comprises one or more of monoclonal antibodies (such as alemtuzumab, atezolizumab, ipilimumab, nivolumab, and pembrolizumab), anti-CD47 antibodies, anti-SIRP-alpha antibodies, anti-GD2 antibodies, anti-PD-1 antibodies, anti-PD-L1 antibodies, immune checkpoint inhibitors (such as CTLA-4 inhibitors), or toll-like receptor (TLR) agonists.

23. The method of claim 1, wherein the cancer comprises metastatic cancer cells or wherein the cancer comprises cells that overexpress CD36 antigen.

24.-72. (canceled)

73. A compound for the treatment of cancer of formula (I)
A¹-X¹-X²-A² (I)
wherein:
A¹ is a carboxylic acid group, a carboxylate anion, or a carboxylate ester;
A² is a taxane moiety, which has a molecular weight of no more than 1600 Da;
X¹ is a hydrophobic group; and
X² is a direct bond, an organic group, —O—, —S—, —S(═O)—, —S(═O)₂—, —S—S—, —N═, ═N—, —N(H)—, —N═N—N(H)—, —N(H)—N═N—, —N(OH)—, or —N(═O)—.

74. The compound of claim 73, wherein A¹ is a carboxylic acid group.

75. The compound of claim 73, wherein the taxane moiety is a paclitaxel moiety or a docetaxel moiety.

76. The compound of claim 75, wherein:
the paclitaxel moiety is a moiety of the formula: 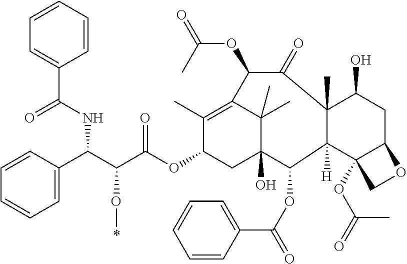 [SMILES: *O[C@@H](C(=O)O[C@H]1C[C@@]2(O)[C@@H](OC(=O)c3ccccc3)[C@@H]3[C@]4(OC(C)=O)CO[C@@H]4C[C@H](O)[C@@]3(C)C(=O)[C@H](OC(C)=O)C(=C1C)C2(C)C)[C@@H](NC(=O)c1ccccc1)c1ccccc1]
or wherein the compound is the compound of formula: 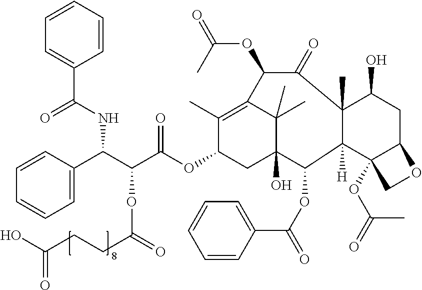
or a pharmaceutically acceptable salt thereof.

77. A pharmaceutical composition comprising an amount of a compound of claim 73 effective for treatment of a cancer which comprises cells that overexpress one or more fatty acid uptake proteins and a pharmaceutically acceptable carrier and which optionally further comprises a protein, wherein the protein is human serum albumin or a protein whose sequence is at least 50% equivalent to that of human serum albumin.
